(19) United States
(12) Patent Application Publication
(10) Pub. No.: US 2012/0183770 A1
(43) Pub. Date: Jul. 19, 2012

(54) MODIFIED CARBON NANOTUBES, METHODS FOR PRODUCTION THEREOF AND PRODUCTS OBTAINED THEREFROM

(76) Inventors: Clive P. Bosnyak, Dripping Springs, TX (US); Kurt W. Swogger, Austin, TX (US)

(21) Appl. No.: 13/164,456

(22) Filed: Jun. 20, 2011

Related U.S. Application Data
(60) Provisional application No. 61/357,420, filed on Jun. 22, 2010.

(51) Int. Cl. H01G 9/035; C07C 51/16; C07C 51/305; C08L 63/00; C08K 3/04; C07C 63/48; C07C 305/02; B82Y 40/00; B82Y 30/00

(52) U.S. Cl. 428/367; 562/488; 562/408; 558/37; 252/62.2; 252/506; 523/468; 977/748; 977/750; 977/752; 977/779; 977/743; 977/847; 977/832

(57) ABSTRACT

The present invention relates to the exfoliation and dispersion of carbon nanotubes resulting in high aspect ratio, surface-modified carbon nanotubes that are readily dispersed in various media. A method is disclosed for their production in high yield. Further modifications by surface active or modifying agents are also disclosed. Application of the carbon nanotubes of this invention as composites with materials such as elastomers, thermosets and thermoplastics are also described.

### CROSS REFERENCE TO RELATED APPLICATIONS

This application claims priority to U.S. Provisional Patent Application No. 61/357,420, filed Jun. 22, 2010, entitled MODIFIED CARBON NANOTUBES, METHODS FOR PRODUCTION THEREOF AND PRODUCTS OBTAINED THEREFROM, the entire content of which is hereby incorporated by reference. This application also incorporates by reference the entire content of each of the following applications: PCT patent application PCT/US09/68781 filed Dec. 18, 2009, which claims priority to U.S. provisional patent applications 61/138,551, filed Dec. 18, 2008, and 61/139,050, filed Dec. 19, 2008.

### BACKGROUND

The present invention relates to the exfoliation and dispersion of carbon nanotubes resulting in high aspect ratio, surface-modified carbon nanotubes that are readily dispersed in various media. Also, the present invention pertains to methods for production of such carbon nanotubes in high yield. These carbon nanotubes are further modified by surface active or modifying agents. This invention also relates to the carbon nanotubes as composites with materials such as elastomers, thermosets and thermoplastics.

Carbon nanotubes in their solid state are currently produced as agglomerated nanotube bundles in a mixture of chiral or non-chiral forms. Various methods have been developed to debundle or disentangle carbon nanotubes in solution. For example, carbon nanotubes may be shortened extensively by aggressive oxidative means and then dispersed as individual nanotubes in dilute solution. These tubes hare low aspect ratios not suitable for high strength composite materials. Carbon nanotubes may also be dispersed in very dilute solution as individuals by sonication in the presence of a surfactant. Illustrative surfactants used for dispersing carbon nanotubes in solution include, for example, sodium dodecyl sulfate and PLURONICS. In some instances, solutions of individualized carbon nanotubes may be prepared from polymer-wrapped carbon nanotubes. Individualized single-wall carbon nanotube solutions have also been prepared in very dilute solutions using polysaccharides, polypeptides, water-soluble polymers, nucleic acids, DNA, polynucleotides, polyimides, and polyvinylpyrrolidone. The dilution ranges are often in the mg/liter ranges and not suitable for commercial usage.

A number of uses for carbon nanotubes have been proposed including, for example, energy storage devices (e.g. ultracapacitors, supercapacitors and batteries), field emitters, conductive films, conductive wires and membrane filters. Use of carbon nanotubes as a reinforcing agent in polymer composites is another area in which carbon nanotubes are predicted to have significant utility. However, utilization of carbon nanotubes in these applications has been hampered due to the general inability to reliably produce individualized carbon nanotubes. For example, load transfer to carbon nanotubes in polymer composites is typically less than would be expected than if the carbon nanotubes were fully exfoliated as individual nanotubes.

Likewise, in applications involving electrical conduction, conductivity is lower than anticipated due to reduced access to the carbon nanotube surface when the carbon nanotubes are agglomerated as opposed to being dispersed as individuals. As noted above, current methods for producing exfoliated carbon nanotubes usually results in severe shortening and/or functionalization of the nanotubes. Without proper individual separation of the carbon nanotubes, it is also likely that non-uniform functionalization of the tube surface results. Such shortening, functionalization or non-uniform functionalization also generally results in reduced conductivity, which is also disadvantageous for applications where high electrical conductivity is beneficial.

In view of the foregoing, solid exfoliated carbon nanotubes and methods for efficiently exfoliating carbon nanotubes are of considerable interest in the art. Such exfoliated carbon nanotubes are likely to exhibit considerably improved properties in applications including, for example, energy storage devices and polymer composites. Further surface modification of the tubes for enhanced bonding to a material or attaching electroactive materials are facilitated by exfoliation. These further surface modified carbon nanotubes are considered advantageous for energy applications such as batteries and capacitors and photovoltaics, and material-composite applications such as tires, adhesives, and engineering composites such as windblades.

### SUMMARY

In various embodiments, a plurality of carbon nanotubes is disclosed comprising single wall, double wall or multiwall carbon nanotube fibers having an aspect ratio (the ratio of length of the nanotube to the diameter of the nanotube) of from about 25 to about 500, preferably from about 60 to about 250, and a oxidation level of from about 3 weight % to about 15 weight %, preferably from about 5 weight % to about 12 weight % and most preferably 6 weight % to about 10 weight % (weight % is the ratio of the weight of a component divided by the total weight expressed as a percentage). Preferably, a neutralized water treatment of the fibers results in a pH of from about 4 to about 9, more preferably from about 6 to about 8. The fibers can have oxidation species comprising of carboxylic acid or derivative carboxylate groups and are essentially discrete individual fibers, not entangled as a mass.

In other embodiments, the fibers comprise a residual metal concentration of less than about 1000 parts per million, ppm, and preferably less than about 100 ppm. The fibers can be open-ended and the matt of fibers has an electrical conductivity of at least 0.1 Siemens/cm and as high as 100 Siemens/cm.

In another embodiment, the fibers can be mixed with a material such as, but not limited to, an elastomer or thermoplastic or thermoset to form a material-carbon nanotube composite.

In yet other embodiments, the fibers have an average diameter of from about 0.6 nanometers to about 30 nanometers, preferably from about 2 nm to about 15 nm and most preferably 6-12 nm. The fibers have an average distribution length of from about 50 nanometers to about 10000 nanometers, preferably from about 400 nm to about 1200 nm.

In another embodiment, a method for preparing carbon nanotube fibers is disclosed, said method comprising suspending entangled non-discrete multi-w all carbon nanotube fibers in an acidic solution, optionally agitating said composition, sonically treating said suspended nanotube fiber composition to form discrete carbon nanotube fibers and isolating the resultant discrete carbon nanotube fibers from the composition using solid-liquid methods such as filtration or centrifugation prior to further treatment.

In another embodiment, the method for preparing carbon nanotube fibers includes an acidic solution which comprises a solution of sulfuric acid and nitric acid wherein the nitric acid is present in a dry basis of from about 10 wt % to about 50 wt %, preferably from about 15 wt % to about 30 wt %.

In another embodiment, the method for preparing carbon nanotube fibers includes the carbon nanotube fibers are present in a concentration of from greater than zero to less than about 4 weight percent of the suspended nanotube fiber composition.

In another embodiment, the method for preparing, carbon nanotube fibers includes wherein the sonic treatment is performed at an energy input of from about 200 to about 600 Joules/gram of suspended composition, preferably from about 250 to about 350 Joules/gram of suspended composition.

In other various embodiments, the method for preparing carbon nanotube fibers includes wherein the suspended discrete nanotube fiber composition in the acidic solution is controlled at a specific temperature environment from about 15 to about 65° C., preferably from about 25 to about 35° C.

In another embodiment, the method for preparing carbon nanotube fibers comprises a batch, semi-batch or continuous method.

In another embodiment, the method for preparing carbon nanotube fibers includes wherein the composition is in contact with the acidic solution from about 1 hour to about 5 hours, preferably from about 2.5 to about 3.5 hours.

In yet another embodiment, the method for preparing carbon nanotube fibers includes wherein said isolated resultant discrete carbon nanotube fibers from the composition prior to further treatment comprises at least about 10 weight percent water.

In another embodiment, the discrete carbon nanotube fibers are made in at least a 30% yield from the initial charge of as-received non-discrete nanotubes with the preferred yield >80%.

In some embodiments, the fibers are at least partially (>5° A) surface modified, or coated, with at least one modifier, or at least one surfactant.

In some embodiments, the fibers are completely (>80%) surface modified, or coated.

In some embodiments, the fibers are at least partially surface modified or coated wherein the surfactant or modifier is hydrogen bonded, covalently bonded, or ionically bonded to the carbon nanotube fibers.

In some embodiments, the completely surface modified or coated fibers include wherein said surface modification or coating is substantially uniform.

In other embodiments, at least partially, or fully surface modified fibers are further mixed or blended with at least one organic or inorganic material to form a material-nanotube fiber composition.

In another embodiment, the material-nanotube fiber composition includes wherein the fiber surface modifier or surfactant is chemically bonded to the material and/or fiber.

In another embodiment, the at least partially, or fully surface modified fibers are further mixed or blended with at least one elastomer to form an elastomer nanotube fiber composition.

In another embodiment, the elastomer nanotube fiber composition includes wherein the fiber surface modifier or surfactant is chemically bonded to the elastomer and/or fiber.

In another embodiment, the elastomer nanotube fiber composition, particularly materials made from elastomers, commonly called either natural or synthetic rubber or rubber compounds which can include fillers such as carbon or silicon compounds, includes wherein the fiber surface modifier or surfactant is chemically or physically (or both) bonded to the elastomer and/or the isolated fibers and/or any fillers present.

In another embodiment, the at least partially, or fully surface modified fibers are further mixed or blended with at least one epoxy to form an epoxy nanotube fiber composition.

In another embodiment, the epoxy nanotube fiber composition includes wherein the fiber surface modifier or surfactant is chemically bonded to the epoxy and/or fiber.

In an additional embodiment, the elastomer nanotube fiber composition has a fatigue crack failure resistance of at least 2 to about 20 times the fatigue crack failure resistance of the elastomer tested without carbon nanotubes.

In an another embodiment, the epoxy nanotube fiber composition has a fatigue crack failure resistance of at least 2 to about 20 times the fatigue crack failure resistance of the epoxy tested without carbon nanotubes.

In another embodiment, the epoxy/nanotube fiber composition has a coefficient of expansion in at least one dimension of at least ⅔ to ⅓ that of the epoxy tested without carbon nanotubes in the same dimension.

In yet other embodiments the material-nanotube fiber composition exhibits superior adhesion or cohesion to a substrate by at least a factor of two compared to the same material without the nanotube tested similarly.

In another embodiment the nanotube fibers are further mixed or blended and/or sonicated with at least one elastomer and an inorganic nanoplate to form an elastomer nanotube fiber and nanoplate composition.

The foregoing has outlined rather broadly various features of the present disclosure in order that the detailed description that follows may be better understood. Additional features and advantages of the disclosure will be described hereinafter, which form the subject of the claims.

In various embodiments, compositions of fully and high aspect ratio exfoliated carbon nanotubes are disclosed herein. The exfoliated carbon nanotubes are dispersed in the solid state such as, for example, a mat of dispersed carbon nanotubes. The exfoliated carbon nanotubes are maintained in a dispersed state without being dispersed in a continuous matrix such as, for example, a polymer matrix dispersant or a solution.

In other various embodiments, methods for preparing exfoliated carbon nanotubes are disclosed herein.

In some embodiments, the methods for preparing exfoliated carbon nanotubes include suspending carbon nanotubes in a solution containing a first quantity of a nanocrystalline material, precipitating a first quantity of exfoliated carbon nanotubes from the solution and isolating the first quantity of exfoliated carbon nanotubes.

In some embodiments, the methods for preparing exfoliated carbon nanotubes include suspending carbon nanotubes in a solution containing hydroxyapatite, precipitating exfoliated carbon nanotubes from the solution and isolating the exfoliated carbon nanotubes.

In some embodiments, the methods for preparing exfoliated carbon nanotubes include suspending carbon nanotubes in a solution containing a nanorod material, precipitating exfoliated carbon nanotubes from the solution and isolating the exfoliated carbon nanotubes.

In some embodiments, the methods for preparing exfoliated carbon nanotubes include preparing a solution of carbon nanotubes in a superacid and filtering the solution through a filter to collect exfoliated carbon nanotubes on the filter.

In still other various embodiments, energy storage devices containing exfoliated carbon nanotubes are disclosed herein. In some embodiments, the energy storage device is a battery containing at least two electrodes and an electrolyte in contact with the at least two electrodes. At least one of the electrodes contains exfoliated carbon nanotubes.

### BRIEF DESCRIPTION OF THE DRAWINGS

For a more complete understanding of the present disclosure, and the advantages thereof, reference is now made to the following descriptions to be taken in conjunction with the accompanying drawings describing specific embodiments of the disclosure, wherein:

FIG. 1 shows an illustrative arrangement of the basic elements of a Faradaic capacitor;

FIG. 2 shows an illustrative arrangement of the basic elements of an electric double layer capacitor;

FIG. 3 shows an illustrative arrangement of the basic elements of a battery;

FIG. 4 shows an illustrative electron micrograph of hydroxyapatite plates having diameters of 3-15 μm;

FIG. 5 shows an illustrative electron micrograph of hydroxyapatite nanorods having lengths of 100-200 nm;

FIG. 6A shows an illustrative electron micrograph of as-received multi-wall carbon nanotubes; FIG. 6B shows an illustrative electron micrograph of multi-wall carbon nanotubes exfoliated using hydroxyapatite nanorods;

FIG. 7A shows an illustrative EDX spectrum of precipitated exfoliated multi-wall carbon nanotubes; FIG. 7B shows an illustrative EDX spectrum of precipitated exfoliated multi-wall carbon nanotubes after acid washing;

FIG. 8 shows an illustrative electron micrograph of exfoliated multi-wall carbon nanotubes after precipitation and washing;

FIG. 9 shows an illustrative electron micrograph of exfoliated carbon nanotubes obtained from 3:1 H₂SO₄:HNO₃;

FIG. 10 shows an illustrative electron micrograph of exfoliated double-wall carbon nanotubes following acid exfoliation and treatment with sodium dodecyl sulfate; and

FIG. 11 shows an illustrative electron micrograph of exfoliated carbon nanotubes decorated with copper oxide nanoparticles.

FIG. 12 shows a thermogravimetric plot of carbon nanotubes of this invention with oxidation species of various levels;

FIG. 13 shows an illustrative fourier transform infra red spectrum of an untreated carbon nanotube and of an oxidized carbon nanotube of this invention in the wavenumber range 2300 to 1300 cm⁻¹;

FIG. 14. Representative engineering stress strain curves for the unfilled and fiber filled SBR; and

FIG. 15. Engineering Stress-engineering strain curves for a polypropylene-ethylene copolymer with 1% wt carbon nanotubes of this invention and without carbon nanotubes.

### DETAILED DESCRIPTION

In the following description, certain details are set forth such as specific quantities, sizes, etc., so as to provide a thorough understanding of the present embodiments disclosed herein. However, it will be evident to those of ordinary skill in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

While most of the terms used herein will be recognizable to those of ordinary skill in the art, it should be understood, however, that when not explicitly defined, terms should be interpreted as adopting a meaning presently accepted by those of ordinary skill in the art. In cases where the construction of a term would render it meaningless or essentially meaningless, the definition should be taken from Webster's Dictionary, 3rd Edition, 2009. Definitions and/or interpretations should not be incorporated from other patent applications, patents, or publications, related or not, unless specifically stated in this specification or if the incorporation is necessary for maintaining validity.

Various embodiments presented hereinbelow reference carbon nanotubes. In particular, in various embodiments, bundled or entangled carbon nanotubes can be debundled or unentangled according to the methods described herein to produce exfoliated carbon nanotube solids. The carbon nanotubes being debundled or unentangled can be made from any known means such as, for example, chemical vapor deposition, laser ablation, and high pressure carbon monoxide synthesis (HiPco). The bundled or entangled carbon nanotubes can be present in a variety of forms including, for example, soot, powder, fibers, and bucky paper. Furthermore, the bundled or entangled carbon nanotubes may be of any length, diameter, or chirality. Carbon nanotubes may be, semi-metallic, semi-conducting or non-metallic based on their chirality and number of walls. In various embodiments, the bundled and/or exfoliated carbon nanotubes may include, for example, single-wall carbon nanotubes (SWNTs), double-wall carbon nanotubes (DNANTs), multi-wall carbon nanotubes (MWNTs), shortened carbon nanotubes, oxidized carbon nanotubes, functionalized carbon nanotubes, and combinations thereof. One of ordinary skill in the art will recognize that many of the specific embodiments referenced hereinbelow utilizing a particular type of carbon nanotube may practiced equivalently within the spirit and scope of the disclosure utilizing other types of carbon nanotubes.

Functionalized carbon nanotubes of the present disclosure generally refer to the chemical modification of any of the carbon nanotube types described hereinabove. Such modifications can involve the nanotube ends, sidewalls, or both. Chemical modifications may include, but are not limited to covalent bonding, ionic bonding, chemisorption, intercalation, surfactant interactions, polymer wrapping, cutting, solvation, and combinations thereof. In some embodiments, the carbon nanotubes may be functionalized before, during and after being exfoliated.

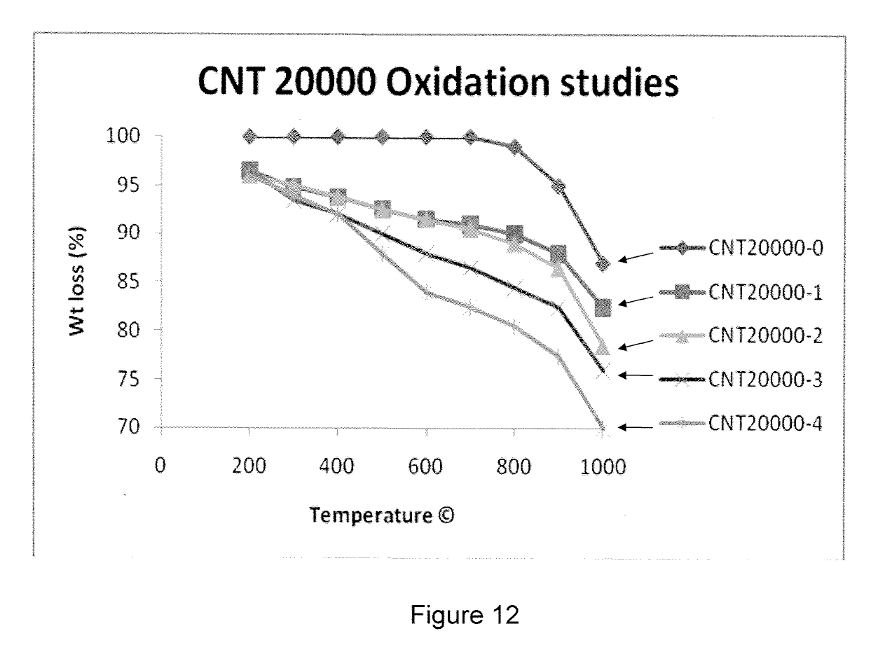

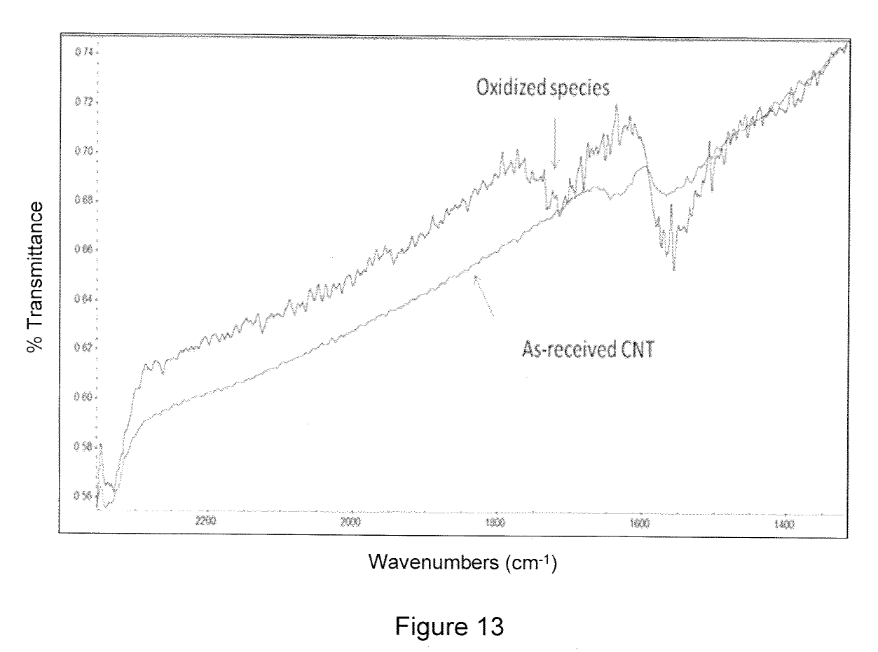

In various embodiments, a plurality of carbon nanotubes is disclosed comprising single wall, double wall or multiwall carbon nanotube fibers having an aspect ratio of from about 25 to about 500, preferably from about 60 to about 200, and a oxidation level of from about 3 weight % to about 15 weight %, preferably from about 5 weight % to about 10 weight %. The oxidation level is defined as the amount by weight of oxygenated species covalently bound to the carbon nanotube. In FIG. 12 is an example of a thermogravimetric plot illustrative of the method of determination of the % weight of oxygenated species on the carbon nanotube. The thermogravimetric method involves taking about 5 mg of the dried oxidized carbon nanotube and heating at 5° C./minute from room temperature to 1000 degrees centigrade in a dry nitrogen atmosphere. The % weight loss from 200 to 600 degrees centigrade is taken as the % weight loss of oxygenated species. The oxygenated species can also be quantified using fourier transform infra-red spectroscopy, FTIR. FIG. 13 and energy-dispersive X-ray (EDX) analyses.

Preferably, a neutralized water treatment of the fibers results in a pH of from about 4 to about 9, more preferably from about 6 to about 8. The pH of the matt of oxidized carbon nanotubes can conveniently adjusted using an alkaline solution such as aqueous ammonium hydroxide. A certain residual time is allowed for the acid or alkaline molecules from the internal regions of the carbon nanotube to diffuse out or in. The fibers can have oxidation species comprising of carboxylic acid or derivative carbonyl containing species and are essentially discrete individual fibers, not entangled as a mass. The derivative carbonyl species can include ketones, quaternary amines, amides, esters, acyl halogens, metal salts and the like.

As-made carbon nanotubes using metal catalysts such as iron, aluminum or cobalt can retain a significant amount of the catalyst associated or entrapped within the carbon nanotube, as much as 5% by weight. These residual metals can be deleterious in such applications as electronic devices because of enhanced corrosion. In other embodiments, the oxidized fibers comprise a residual metal concentration of less than about 1000 parts per million, ppm, and preferably less than about 100 ppm. The metals can be conveniently determined using EDX.

In another embodiment, the fibers can be open-ended to allow transport or storage of small molecules such as ethane or propane.

In yet another embodiment, the matt of fibers has an electrical conductivity of at least 0.1 Siemens/cm and as high as 100 Siemens/cm. A convenient measurement of conductivity is made using a digital ohmmeter with copper strips 1 cm apart on a matt of the fibers compressed with hand pressure between two polystyrene discs.

In another embodiment, the fibers can be mixed with an organic or inorganic material to form a material-carbon nanotube composite. Organic materials can include such as, but not limited to, an elastomer, thermoplastic or thermoset or combinations thereof. Examples of elastomers include, but not limited to polybutadiene, polyisoprene, polystyrene-butadiene, silicones, poly urethanes, polyolefins and polyether-esters. Examples of thermoplastics include amorphous thermoplastics such as polystyrenics, polyacrylates, and polycarbonates, and semi-crystalline thermoplastics such as polyolefins, polypropylene, polyethylene, poly amides, polyesters, and the like. The exfoliated carbon nanotube fibers of this invention impart significant strength and stiffness to the materials even at low loadings. These new elastomer nanotube filler materials can improve or affect the frictional, adhesive, cohesive, noise and vibration, rolling resistance, tear, wear fatigue and crack resistance, hysteresis, large strain effects (Mullins effect), small strain effects (Payne effect) and oscillation or frequency properties and swelling resistance to oil of the elastomers and elastomer compounds. This change in properties will be beneficial for applications such as tires or other fabricated rubber or rubber compounded parts.

In yet other embodiments, the carbon nanotube fibers have an average diameter of from about 0.6 nanometers to about 30 nanometers, preferably from about 2 nm to about 15 nm and most preferably 6-12 nm. Single wall carbon nanotubes have diameters as low as 0.6 nm and the interwall dimension is about 0.34 nm. The fibers have a length of from about 50 nanometers to about 10.000 nanometers, preferably from about 400 nm to about 1,200 nm.

In other embodiments, a method for preparing carbon nanotube fibers is disclosed of suspending entangled non-discrete multi-wall carbon nanotube fibers in an acidic solution over time, optionally agitating said composition, while sonically treating the suspended nanotube fiber composition to form discrete carbon nanotube fibers and isolating the resultant discrete carbon nanotube fibers from the composition using solid/liquid separation such as filtration or centrifugation prior to further treatment. The acidic solution comprises a mixture of sulfuric acid and nitric acid where the nitric acid is present in a dry basis of from about 10 wt % to about 50 wt %, preferably from about 15 wt % to about 30 wt. %. The method also includes the carbon nanotube fibers being present in a concentration of from greater than zero to less than about 4 weight percent of the suspended nanotube fiber composition with a preference of 1 to 2%. Above about 2% by weight the carbon nanotubes interact with each other such that the viscosity rises rapidly and stirring and sonication can become non-uniform, which can result in non-uniform oxidation of the fibers.

In another embodiment, the method for preparing carbon nanotube fibers includes wherein the sonic treatment is performed at an energy input of from about 200 to about 600 Joules/gram of suspended composition, preferably from about 250 to about 350 Joules/gram of suspended composition. If there is a large excess of sonic energy much above about 600 joules/gram of suspended composition this excess energy can lead to the fibers being damaged and too short in length for optimum performance in applications such as material-fiber composites.

In other various embodiments, the method for preparing carbon nanotube fibers includes wherein the suspended nanotube fiber composition in the acidic solution is controlled at a specific temperature environment from about 15 to 65° C., preferably from about 25 to about 35° C. Above about 65° C. in the acid medium, the rate of oxidation is very rapid and not well-controlled leading to severe degradation of the tube length and great difficulty in filtering the fibers. Below about 15° C. the rate of oxidation can be too slow for economic production of the fibers.

In another embodiment, the method for preparing carbon nanotube fibers comprises a batch, semi-batch or continuous method. The continuous method can involve using temperature controlled sonicator cells in conjunction with circulating pumps with different energy inputs and a centrifuge for filtering and washing of the exfoliated carbon nanotube product.

In other embodiments, the method for preparing carbon nanotube fibers includes wherein the composition is in contact with the acidic solution from about 1 hour to about 5 hours, preferably from about 2.5 to about 3.5 hours. The choice of the time and temperature interval are given by the degree of oxidation of the exfoliated carbon nanotubes required for the end-use application After isolated resultant discrete carbon nanotube fibers from the acid composition prior to further treatment the mats can contain at least about 10 weight percent water. This method facilitates the subsequent exfoliation in other materials. The discrete carbon nanotube fibers are made in at least a 30% yield from the initial charge of nanotubes as-received with the preferred yield >80%.

### Example 1

An illustrative process for producing oxidized carbon nanotubes follows: 3 liters of sulfuric acid, 97% sulfuric acid and 3% water, and 1 liter of concentrated nitric acid containing 70% nitric acid and 30% water, are added into a 10 liter temperature controlled reaction vessel fitted with a sonicator and stirrer. 400 grams of non-discrete carbon nanotubes, grade Flowtube 9000 from CNano corporation, are loaded into the reactor vessel while stirring the acid mixture and the temperature maintained at 25° C. The sonicator power is set at 130-150 watts and the reaction is continued for 3 hours. After 3 hours the viscous solution is transferred to a filter with a 5 micron filter mesh and much of the acid mixture removed by filtering using a 100 psi pressure. The filter cake is washed 1 times with 4 liters of deionized water followed by 1 wash of 4 liters of an ammonium hydroxide solution at pH>9 and then 2 more washes with 4 liters of deionized water. The resultant pH of the final wash is >4.5. A small sample of the filter cake is dried in vacuo at 100° C. for 4 hours and a thermogravimetric analysis taken as described previously. The amount of oxidized species on the fiber is 8% weight.

### Example 2

An example of the control of carbon nanotube oxidation for a different carbon nanotube grade. Flowtube 20000 is given in FIG. 12 which shows the weight loss of Flowtube 20000 at various times in contact with an acid mixture at 25° C. and after being separated from the acid mixture, washed with deionized water and dried.

In some embodiments, the fibers are at least partially or fully surface modified or coated with at least one modifier or at least one surfactant. The surface modifier or coating or surfactant is hydrogen bonded, covalently bonded, or ionically bonded to the carbon nanotube fibers. Suitable surfactants include, but are not limited, to both ionic and non-ionic surfactants, sodium dodecyl sulfate, sodium dodecylbenzene sulfonate, and PLURONICS. Cationic surfactants are chiefly used for dispersion in non-polar media, such as, for example, chloroform and toluene. Other types of molecules such as, for example, cyclodextrins, polysaccharides, polypeptides, water soluble polymers, DNA, nucleic acids, polynucleotides, and polymers such as polyimides and polyvinyl pyrrolidone, can be used to redisperse the oxidized carbon nanotubes. Furthermore, the surface modification or coating can be substantially uniform.

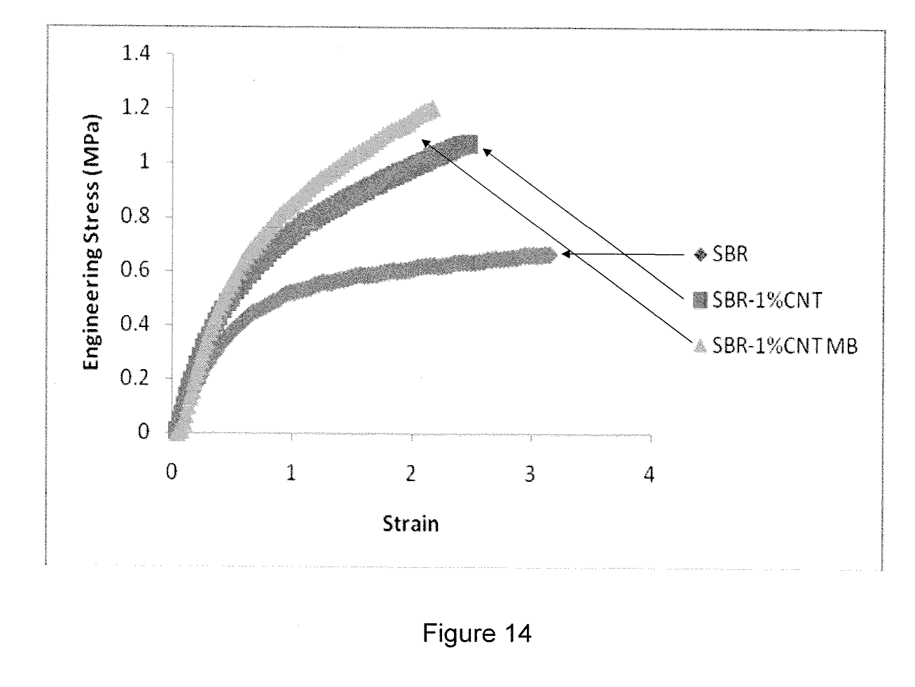

In other embodiments, at least partially, or fully surface modified fibers are further mixed or blended and/or sonicated with at least one organic or inorganic material to form a material-nanotube fiber composition. As illustrative examples carbon nanotubes are oxidized to a level of 8% weight, with an average tube diameter of 12 nm and average length 600 nm and mixed into various materials. In one example the 1% weight fiber is mixed with commercial styrene-butadiene, SBR, polymer obtained from Goodyear. This is labeled SBR 1% MWNT in Table 1. In another approach, a master-batch, MB, is made of a concentrate of SBR and 10% weight fiber, followed by melt mixing with more SBR to give 1% weight fiber content. This is labeled SBR 1% MWNT MB in FIG. 14 and in Table 1. A control of SBR without fiber is made under exactly the same thermal history and with the same curing package. The curing package contains zinc oxide, stearic acid, sulfur and t-butyl benzothiazole sulfonamide.

After curing the films are tested in tension at 25° C. using a tensile tester with an initial strain rate of 1×10⁻² s⁻¹ at 25° C. Tensile modulus is the ratio of engineering stress to strain at the beginning of the tensile test. Engineering Stress is the load divided by the initial cross-sectional area of the specimen. Strain is defined as the distance traversed by the crosshead of the instrument divided by the initial distance between the grips.

TABLE 1
Tensile properties of cured SBR and SBR with MWNT.

| | Average | Average | Average Strain |
|---|---|---|---|
| Material | Modulus (MPa) | Strength (MPa) | to Break |
| SBR | 1.22 | 0.64 | 2.8 |
| SBR + 1% MWNT | 1.58 | 0.94 | 2.4 |
| SBR + 1% MWNT | 1.63 | 0.97 | 1.7 |
| MB | | | |

A 30% increase in the values of tensile modulus and 50% increase in the tensile strength are gained using 1% weight of the oxidized carbon nanotubes of this invention. These attributes are important elements that will lead to improved wear.

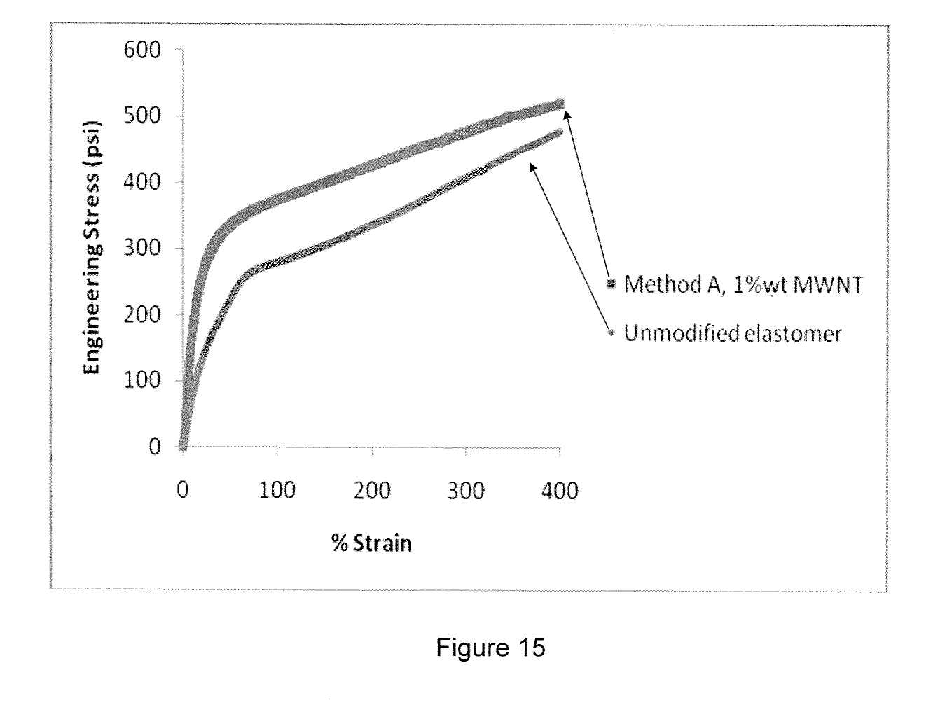

Using another, elastomer, in this case a semi-crystalline propylene-ethylene copolymer, Versify resins from The Dow Chemical Co, after melt mixing and solidification the elastomer containing 1% weight modified tubes gave an improvement in strength of about 50%, see FIG. 15.

In another embodiment, the elastomer nanotube fiber composition, particularly materials made from elastomers commonly called either natural or synthetic rubber or rubber compounds (with the addition of tillers such as carbon or silicon) includes wherein the fiber surface modifier or surfactant is chemically or physically (or both) bonded to the elastomer and/or the isolated fibers or the tiller in the compounds.

In another embodiment, the material-nanotube fiber composition includes wherein the fiber surface modifier or surfactant is chemically bonded to the material and/or fiber. As an example, oleylamine (1-amino-9-octadecene) can be reacted with carbon nanotubes containing carboxylic groups to give the amide. On addition of the amide modified carbon nanotube fiber to a vinyl containing polymer material such as styrene-butadiene followed by addition of crosslinking agents comprising such as peroxides or sulfur, the vinyl containing polymer can be covalently bonded to the amide functionality of the carbon nanotube.

In another embodiment, the at least partially, or fully surface modified fibers are further mixed or blended with at least one epoxy to form an epoxy nanotube fiber composition. In this example the oxidized carbon nanotubes are dispersed in a bisphenol F epoxy at high temperature using a sonicator and mechanical mixer. The epoxy is cured at 110° C. for 2 hours using tetraethylene tetraamine. Results of tensile testing are shown in Table 2.

TABLE 2

| Sample | % CNT | Modulus psi | Stress bk | Elong Bk % |
|---|---|---|---|---|
| Bisphenol F | 0 | 152330 | 10040 | 10.7 |
| Bisphenol F + | 0.4 | 194190 | 12275 | 12 |
| 0.4% wt fiber | | | | |

The fatigue properties of the material-carbon fiber composites of this invention also show a fatigue crack failure resistance of at least 2 to about 20 times the fatigue crack failure resistance of the material tested without carbon nanotubes. A usual test procedure for fatigue crack failure resistance is to take a dogbone specimen and introduce a razor notch 1/10 the width of the specimen in the center of the length of the specimen. The specimen is subjected to oscillation with a maximum stress less than the yield stress determined under monotonic loading to break. The number of cycles to break under a given loading history is recorded.

In another embodiment, the epoxy/nanotube fiber composition has a coefficient of expansion in at least one dimension of at least ⅔ to ⅓ that of the epoxy tested without carbon nanotubes in the same dimension. As an illustrative example follows: ERL 4221, a cycloaliphatic epoxy resin, Dow Chemical Co., is mixed with 1% weight of the oxidized fiber of this invention. It was then mixed and cured with an anhydride ECA 100, Dow Chemical Co., at 180° C. for 2 hours. The plaque gave a through thickness linear thermal coefficient of expansion of 4.5×10⁻⁵ m/m/° C. compared to a control similarly cured, but with no carbon nanotube fiber which gave a value of 8.4×10⁻⁵ m/m/° C.

In another embodiment the nanotube fibers are further mixed or blended and/or sonicated with at least one material and an inorganic nanoplate to form a material nanotube fiber and nanoplate composition. The materials can be elastomers, thermoplastics and thermosets. The nanoplates can be, for example, clays, transition metal containing phosphates or graphene structures. The nanoplates have an individual plate thickness less than 20 nm. The nanotube fibers of this invention can disperse between the individual nanoplates.

The oxidized and exfoliated carbon nanotubes of the present disclosure take advantage of physical properties offered by individual carbon nanotubes that are not apparent when the carbon nanotubes are aggregated into bundles. For example, in various embodiments, the oxidized and exfoliated carbon nanotubes may be advantageously used in a wide range of applications including capacitors, batteries, photovoltaics, sensors, membranes, static dissipators, electromagnetic shields, video displays, pharmaceuticals and medical devices, polymer composites, various adhesives, and gas storage vessels. In various embodiments, the oxidized and exfoliated carbon nanotubes may also be used in fabrication and assembly techniques including, for example, ink-jet printing, spraying, coating, melt extruding, thermoforming, blow-molding, film blowing, foaming and injection molding.

### Additional Examples

Various embodiments presented herein below reference carbon nanotubes. In particular, in various embodiments, bundled carbon nanotubes can be debundled according to the methods described herein to produce exfoliated carbon nanotube solids. The carbon nanotubes being debundled can be made from any known means such as, for example, chemical vapor deposition, laser ablation, and high pressure carbon monoxide synthesis (HiPco). The bundled carbon nanotubes can be present in a variety of forms including, for example, soot, powder, fibers, and bucky paper. Furthermore, the bundled carbon nanotubes may be of any length, diameter, or chirality. Carbon nanotubes may be metallic, semi-metallic, semi-conducting or non-metallic based on their chirality and number of walls. In various embodiments, the bundled and/or exfoliated carbon nanotubes may include, for example, single-wall carbon nanotubes (SWNTs), double-wall carbon nanotubes (DWNTs), multi-wall carbon nanotubes (MWNTs), shortened carbon nanotubes, oxidized carbon nanotubes, functionalized carbon nanotubes, and combinations thereof. One of ordinary skill in the art will recognize that any of the specific embodiments referenced hereinbelow utilizing a particular type of carbon nanotube may practiced equivalently within the spirit and scope of the disclosure utilizing other types of carbon nanotubes.

Functionalized carbon nanotubes of the present disclosure generally refer to the chemical modification of any of the carbon nanotube types described hereinabove. Such modifications can involve the nanotube ends, sidewalls, or both. Chemical modifications may include, but are not limited to covalent bonding, ionic bonding, chemisorption, intercalation, surfactant interactions, polymer wrapping, cutting, solvation, and combinations thereof. In some embodiments, the carbon nanotubes may be functionalized before being exfoliated. In other embodiments, the carbon nanotubes are functionalized after being exfoliated.

In some embodiments, the carbon nanotubes may be further associated or functionalized with an electroactive material. In some embodiments, an electroactive material may be oxides of transition metals such as, for example, Ru, Ir, W, Mo, Mn, Ni and Co. In some embodiments, the electroactive material may be a conducting polymer such as, for example, polyaniline, polyvinylpyrrole or polyacetylene. In some embodiments, the electroactive material may be a nanoparticle or plurality of nanoparticles bound to the carbon nanotubes. For example, in some embodiments, an electroactive nanoparticle may include materials such as SnO₂, Li₄Ti₅O₁₂, silicon nanotubes, silicon nanoparticles and various combinations thereof. Carbon nanotubes associated or functionalized with an electroactive material may be particularly advantageous for applications involving electrical conductivity.

Any of the embodiments herein referencing carbon nanotubes may also be modified within the spirit and scope of the disclosure to substitute other tubular nanostructures, including, for example, inorganic or mineral nanotubes. Inorganic or mineral nanotubes include, for example, silicon nanotubes, boron nitride nanotubes and carbon nanotubes having heteroatom substitution in the nanotube structure. In various embodiments, the nanotubes may include elements such as, for example, carbon, silicon, boron and nitrogen. In further embodiments, the inorganic or mineral nanotubes may also include metallic and non-metallic elements. For example, in some embodiments, the inorganic or mineral nanotubes can be associated with metals, organic compounds, and inorganic compounds. Association may be on the interior or exterior of the inorganic or mineral nanotubes. Exterior association may be a physical association, such as, for example, van der Waals association. Exterior association of these materials may also include either ionic or covalent bonding to the nanotube exterior.

In various embodiments, the present disclosure describes compositions containing exfoliated carbon nanotubes. The exfoliated carbon nanotubes are not dispersed in a continuous matrix that maintains the carbon nanotubes in an exfoliated state. Illustrative continuous matrices include, for example, a solution or a polymer matrix that maintains the carbon nanotubes in at least a partially or substantially exfoliated state. In various embodiments, the exfoliated carbon nanotubes comprise a carbon nanotube mat. As such, the exfoliated carbon nanotubes of the present disclosure are distinguished over exfoliated carbon nanotubes presently known in the art, which may re-agglomerate once removed from solution.

The exfoliated carbon nanotubes of the present disclosure take advantage of physical properties offered by individual carbon nanotubes that are not apparent when the carbon nanotubes are aggregated into bundles. For example, in various embodiments, the exfoliated carbon nanotubes may be advantageously used in a wide range of applications including capacitors, batteries, photovoltaics, sensors, membranes, static dissipators, electromagnetic shields, video displays, pharmaceuticals and medical devices, polymer composites and gas storage vessels. In various embodiments, the exfoliated carbon nanotubes may also be used in fabrication and assembly techniques including, for example, ink-jet printing, spraying, coating, melt extruding, thermoforming, blow-molding and injection molding.

In various embodiments, the exfoliated carbon nanotubes may be single-wall carbon nanotubes, double-wall carbon nanotubes, multi-wall carbon nanotubes and various combinations thereof. In some embodiments, the carbon nanotubes are full-length carbon nanotubes.

In some embodiments, the carbon nanotubes are substantially free of catalytic residues, non-nanotube carbon and various combination thereof. In some embodiments, the carbon nanotubes are purified to remove catalytic residues and non-nanotube carbon. Such purification may take place either before or after the exfoliation of the carbon nanotubes takes place.

In various embodiments, exfoliated carbon nanotubes general y have a diameter of between about 0.7 nm and about 20 nm. Single-wall carbon nanotubes are generally about 0.7 nm to about 10 nm in diameter, whereas multi-wall nanotubes are generally greater than about 10 nm in diameter and up to about 100 nm in diameter in some embodiments. In some embodiments, the exfoliated carbon nanotubes have a diameter between about 1 nm and about 10 nm. In some embodiments, the exfoliated carbon nanotubes have a diameter between about 10 nm and about 100 nm.

The carbon nanotube length varies between about 500 nm and about 10 mm in some embodiments, between about 500 nm and 1 mm in some embodiments, between about 500 nm and 500 μm in some embodiments, between about 500 nm and 1 μm in some embodiments and various subranges thereof. In some embodiments, the exfoliated carbon nanotubes have an average length that is not substantially different than that of the bundled carbon nanotubes from which they are produced. That is, in some embodiments, the carbon nanotubes are full length carbon nanotubes that are not shortened during exfoliation. In some embodiments, the exfoliated carbon nanotubes are prepared from bundled carbon nanotubes, and the exfoliated carbon nanotubes have a narrower distribution of lengths than do the bundled carbon nanotubes. That is, a subrange of exfoliated carbon nanotube lengths may be obtained from a population of bundled carbon nanotubes having a distribution of lengths.

The carbon nanotubes have a length to diameter ratio (aspect ratio) of least about 60 in some embodiments and at least about 100 in other embodiments. In some embodiments, the exfoliated carbon nanotubes are prepared from bundled carbon nanotubes, and the exfoliated carbon nanotubes have a narrower distribution of diameters than do the bundled carbon nanotubes. That is, a subrange of exfoliated carbon nanotube diameters may be obtained from a population of bundled carbon nanotubes having a distribution of diameters.

In various embodiments, the exfoliated carbon nanotubes are further separated by chirality. For example, in the process of exfoliating bundled carbon nanotubes, exfoliated carbon nanotubes of a specific chirality or range of chiral forms may be produced. For example, in some embodiments, the exfoliated carbon nanotubes produced may be metallic, semi-metallic or semiconducting.

In some embodiments, the exfoliated carbon nanotubes are further functionalized. Functionalization may take place either before or after exfoliation. However, Applicants envision that functionalization after exfoliation may be advantageous to take advantage of the greater surface area available in the exfoliated carbon nanotubes compared to their bundled counterparts. In some embodiments, the exfoliated carbon nanotubes are functionalized to include an electroactive material bound to the carbon nanotubes, as set forth in more detail hereinabove.

In some embodiments, the methods for preparing exfoliated carbon nanotubes include suspending carbon nanotubes in a solution containing a first quantity of a nanocrystalline material, precipitating a first quantity of exfoliated carbon nanotubes from the solution and isolating the first quantity of exfoliated carbon nanotubes.

In some embodiments, the methods for preparing exfoliated carbon nanotubes include suspending carbon nanotubes in a solution containing hydroxyapatite, precipitating exfoliated carbon nanotubes from the solution and isolating the exfoliated carbon nanotubes.

In some embodiments, the methods for preparing exfoliated carbon nanotubes include suspending carbon nanotubes in a solution containing a nanorod material, precipitating exfoliated carbon nanotubes from the solution and isolating the exfoliated carbon nanotubes.

In some embodiments of the methods, the carbon nanotubes may be further oriented in an alignment step after isolating the exfoliated carbon nanotubes. In some embodiments, the exfoliated carbon nanotubes may be shaped into a form such as, for example, a mat, film, fiber, cloth, non-woven fabric or felt.

An illustrative process for exfoliating carbon nanotubes follows. Carbon nanotubes can be effectively exfoliated using nanoplate of zirconium phosphate treated with a surfactant such as t-butylammonium hydroxide. The carbon nanotubes and the nanoplates are sonicated for short times to obtain full exfoliation of the carbon nanotubes in aqueous media. By controlling the ionic strength of the mixture after sonication, exfoliated carbon nanotubes can be obtained by simple separation techniques such as, for example, centrifugation. The carbon nanotubes after centrifuging and separating exist in a disordered but non-aggregated state and can easily be resuspended with other surfactant addition. Suitable surfactants for resuspension include, for example, both ionic and non-ionic surfactants, such as, for example, polyvinyl pyrrolidone, sodium dodecyl sulfate and PLURONICS. Cationic surfactants may be used for dispersion in non-polar media, such as chloroform and toluene. Application of an electric potential to the suspension may be used alternatively to or in combination with adjusting the ionic strength.

Although the above process may be used to cleanly separate single-wall carbon nanotubes, multi-wall carbon nanotubes and particularly oxidized multi-wall carbon nanotubes may not be separated as cleanly due to their broader range of ionic potentials. As a result, it is difficult to achieve separation of zirconium phosphate from the exfoliated carbon nanotubes when multi-wall carbon nanotubes are used. Furthermore, zirconium phosphate is particularly difficult to dissolve in acids (solubility=0.12 mg/L in 6 M HCl), and it cannot typically be removed by simple acid washing even after isolating the exfoliated carbon nanotubes.

In various embodiments, the methods for preparing exfoliated carbon nanotubes further include utilizing a solution that contains both a surfactant and a quantity of a nanocrystalline material. Surfactants are well known in the carbon nanotube art to aid in solubilization. Without being bound by theory or mechanism, Applicants believe that when a surfactant is used in preparing exfoliated carbon nanotubes, the surfactant may aid in the initial solubilization or suspension of the carbon nanotubes. Precipitation of exfoliated carbon nanotubes takes place thereafter. In various embodiments of the present disclosure, the surfactant may include, for example, sodium dodecyl sulfate, sodium dodecylbenzene sulfonate, or tetraalkylammonium hydroxide. In some embodiments, the surfactant may also modify the surface of the nanocrystalline material used for exfoliating the carbon nanotubes.

In general, exfoliated carbon nanotubes are prepared according to embodiments of the present disclosure by precipitating exfoliated carbon nanotubes from a solution containing a nanocrystalline material. In some embodiments, the ionic strength of the solution is adjusted to induce precipitation of exfoliated carbon nanotubes. In some embodiments, the electrical potential of the solution is adjusted to induce precipitation of exfoliated carbon nanotubes. In some embodiments, the pH of the solution is adjusted to induce precipitation of exfoliated carbon nanotubes.

In some embodiments, the methods for exfoliating carbon nanotubes include adding a release species to the carbon nanotube suspension to adjust the ionic strength and precipitate exfoliated carbon nanotubes. In some embodiments, the ionic strength can be adjusted with an ionic species such as, for example, a solution of KCl. Although one of ordinary skill in the art will recognize the benefits of using an ionic species for adjustment of ionic strength, non-ionic species such as organic compounds may be used for ionic strength adjustment as well. In some embodiments, an electromagnetic field can be applied to the suspension of exfoliated carbon nanotubes in lieu of or in combination with adjustment of the ionic strength with a release species to induce precipitation of the exfoliated carbon nanotubes. Release species may be organic or inorganic compounds.

After precipitation, exfoliated carbon nanotubes can be isolated by simple separation techniques such as, for example, centrifuging, filtering or settling. The separated, exfoliated carbon nanotubes exist in a disordered but non-aggregated state and can be easily redispersed in various media such as, for example, a liquid or polymer melt. In some embodiments, the redispersion may be aided by addition of a surfactant. Suitable surfactants include, but are not limited, to both ionic and non-ionic surfactants, sodium dodecyl sulfate, sodium dodecylbenzene sulfonate, and PLURONICS. Cationic surfactants are chiefly used for dispersion in non-polar media, such as, for example, chloroform and toluene. As noted above, other types of molecules such as, for example, cyclodextrins, saccharides, polypeptides, water soluble polymers. DNA, nucleic acids, polynucleotides, and polymers such as polyimides and polyvinyl pyrrolidone, can be used to redisperse the exfoliated carbon nanotubes in some embodiments.

In some embodiments, a second quantity of exfoliated carbon nanotubes may be precipitated from the suspension of carbon nanotubes. For example, in an embodiment, adding a second quantity of nanocrystalline material to the suspension results in precipitation of a second quantity of exfoliated carbon nanotubes. In some embodiments, the first quantity of carbon nanotubes and the second quantity of carbon nanotubes have different properties from one another such as, for example, different average lengths, diameters or chiralities. Repeated precipitation of carbon nanotube fractions may be repeated as many times as desired.

In some embodiments, the methods further include removing residual nanocrystalline material from the exfoliated carbon nanotubes. In some embodiments, the carbon nanotubes remain exfoliated after removing the nanocrystalline material. Hence, once the carbon nanotubes become fully exfoliated, they are no longer prone to becoming bundled. In some embodiments, the nanocrystalline material may be removed by washing the exfoliated carbon nanotubes. In some embodiments, the carbon nanotubes may be washed with an acid to remove the nanocrystalline material.

The redispersibility of the carbon nanotubes after removal of the nanocrystalline material may be controlled by changing the surfactant concentration and the rate of addition of the release species. Hence, the redispersibility may be controlled by changing the rate of precipitation of exfoliated carbon nanotubes. In other words, in some embodiments the kinetic rate of carbon nanotube precipitation influences the rate of their redissolution following removal of the nanocrystalline material.

In various embodiments of the present disclosure, carbon nanotubes are exfoliated from bundles of carbon nanotubes using a nanocrystalline material having a crystalline form such as, for example, nanorods, nanoplates, or nanowhiskers, to intersperse between individual carbon nanotubes with addition of energy such as sonification. Nanorods include any inorganic or organic compound that may be induced to crystallize in a rod-like crystalline form. Nanowhiskers include any inorganic or organic compound that may be induced to crystallize in a whisker-like crystalline form. In various embodiments, the nanocrystalline material may include, for example, clays, graphite, inorganic crystalline materials, organic crystalline materials and various combinations thereof.

In some embodiments, the methods for preparing exfoliated carbon nanotubes include suspending carbon nanotubes in a solution containing hydroxyapatite, precipitating exfoliated carbon nanotubes from the solution and isolating the exfoliated carbon nanotubes with subsequent treatment.

In various embodiments, the nanocrystalline material may be, for example, hydroxyapatite and hydroxyapatite derivatives. Hydroxyapaptite derivatives include, for example, fluorapatite. In some embodiments, the hydroxyapatite has a crystalline form such as, for example, nanorods, nanoplates and nanowhiskers. In some embodiments, the methods further include removing the hydroxyapatite from the exfoliated carbon nanotubes. In some embodiments, removing can be accomplished, for example, through washing the exfoliated carbon nanotubes with an acid after their being isolated.

Various sizes of the nanocrystalline material may be used to exfoliate the carbon nanotubes. In some embodiments, the nanocrystalline material may be equal to or larger in size than the longest carbon nanotube present in the sample before exfoliation. In such embodiments, the exfoliated carbon nanotubes can be obtained in discrete fractions following addition of a release species such as, for example, KCl. In other embodiments, the nanocrystalline material has a size that is equal to or less than the longest carbon nanotube present in the sample before exfoliation. In this case, carbon nanotubes equal to or less than the size of the nanocrystalline material may be separated from the carbon nanotube suspension. In various embodiments, larger or smaller sizes of nanocrystalline material can be added to the carbon nanotube suspension to exfoliate carbon nanotube fractions having various carbon nanotube sizes.

In various embodiments, the exfoliated carbon nanotubes are further purified to remove impurities such as, for example, residual metal catalyst and non-nanotube carbon residue. With exfoliated carbon nanotubes, further purification is more easily conducted than like purifications conducted on bundled carbon nanotubes due to the comparatively greater surface area present in the exfoliated carbon nanotubes. Purification techniques include conventional techniques such as, for example, oxidation at elevated temperature (e.g., about 200° C. to about 400° C.) or acid extraction to remove metallic impurities. Illustrative acids that may be used to extract metallic impurities from the exfoliated carbon nanotubes include, for example, various concentrations of hydrochloric, hydrobromic, nitric, chlorosulfonic and phosphoric acids and various combinations thereof. In general, the acid and impurities are removed from the exfoliated carbon nanotubes by rinsing with water, organic solvents or combinations thereof. In some embodiments, supercritical fluids such as, for example, highly compressed CO, or hydrocarbons such as, for example, propane or butane, may also be employed to remove impurities from the exfoliated carbon nanotubes.

In various embodiments, the methods for producing exfoliated carbon nanotubes further include derivatization of the exfoliated carbon nanotubes with at least one functional group. Derivatization may occur either before or after exfoliation has occurred. Numerous methods to derivatize carbon nanotubes are known to those of ordinary skill in the art. For example, diazonium chemistry can be utilized to introduce alkyl or aryl groups, either of which may bear further functionalization, on to the carbon nanotubes. In additional embodiments, treating nanotubes with lithium in liquid ammonia, followed by reaction with an alkyl halide may be used to alkylate carbon nanotubes. Reaction of fluorinated carbon nanotubes with ammonia or amines in the presence of a catalyst such as, for example, pyridine, may be used to functionalize the nanotubes through amine-bearing functionalities. Likewise, fluorinated carbon nanotubes may be functionalized with hydroxyl-containing moieties, which may be functionalized to bear an ether linkage OR, wherein R may be any combination of alkyl, aryl, acyl, and arylacyl groups. Furthermore, R may be further functionalized, for example, with halogens, thiols, amino groups and other common organic functionalities. In addition, the carbon nanotubes may be directly functionalized with thiols, alkyl substituted thiols, aryl substituted thiols, and halogens.

In some embodiments, the first quantity or second quantity of exfoliated carbon nanotubes are selectively precipitated by a physical property such as, for example, chirality, diameter or length. In various embodiments carbon nanotubes are exfoliated using a nanocrystalline material in the form of nanoplates and then further separated by chirality, nanotube length, or nanotube diameter. In various embodiments, carbon nanotubes are exfoliated using a nanocrystalline material in the form of nanorods and then further separated by chirality, nanotube length, or nanotube diameter. In various embodiments, carbon nanotubes are exfoliated using a nanocrystalline material in the form of nanowhiskers and then further separated by chirality, nanotube length, or nanotube diameter. Regardless of how the exfoliated carbon nanotubes are prepared, separation by chirality, length or diameter may be more facile after the carbon nanotubes are isolated.

In some embodiments, a direct separation of carbon nanotubes by chirality, length or diameter may be accomplished by choice of the nanocrystalline material in combination with additional agents. For example, using a nanocrystalline material either alone or in combination with chiral surfactants and/or polymers may allow exfoliated carbon nanotubes to be separated based on length, diameter, chirality, type, and functionality such as, for example, oxidation state and/or defect structure.

In some embodiments, the suspension of carbon nanotubes further includes a chiral agent, resulting in selective precipitation of exfoliated carbon nanotubes by chirality. Chiral agents include, for example, surfactants, polymers and combinations thereof. Chiral agents include molecules such as, for example, R,R-tartaric acid, which have been useful for separation of enantiomeric drugs in electrokinetic chromatography, and enantiomers of polylactic acid. In some embodiments, the chiral agents may be used to separate exfoliated carbon nanotubes of a single chirality or a limited number of chiral configurations from a mixture of carbon nanotubes containing a range of carbon nanotube chiralities. In some embodiments, the chiral agent may be a surfactant that both helps disperse the carbon nanotubes and facilitates the chiral separation. The chiral agent may be associated with or chemically bound to the carbon nanotube surface. In some embodiments, carbon nanotubes separated by chirality also are separated by electronic type (i.e., metallic, semi-metallic and semiconducting).

By using polymers and/or surfactants having a defined chirality, separated populations of exfoliated metallic, semi-metallic, or semi-conducting carbon nanotubes can be obtained. Without being bound by mechanism or theory. Applicants believe that polymers and/or surfactants of defined chirality preferentially wrap a carbon nanotube of a complementary chirality type. By selective carbon nanotube precipitation as described hereinabove, carbon nanotubes may be separated by chirality. Selective carbon nanotube precipitation may occur either in the presence or absence of a nanocrystalline material. Separation techniques such as, for example, solvent/non-solvent addition, co-surfactant addition, and differential temperature gradients may be used to selectively precipitate a chiral population of carbon nanotubes. In various embodiments, the chiral polymers and/or surfactants may be mixtures of tactic molecules. By using tactic polymers with a low thermal degradation temperature such as, for example, polypropylene carbonate, the isolated, exfoliated carbon nanotubes can be easily recovered by thermal degradation of the polymer. For example, polypropylene carbonate can be thermally degraded at less than about 300° C. without damaging carbon nanotubes. In further embodiments, the tactic molecules may be a mixture dissolved in a hydrocarbon solvent such as, for example, toluene or decalin. Illustrative tactic polymers include, for example, atactic polystyrene, iostactic polystyrene, syndiotactic polystyrene, d and l polylactic acid, d and l polypropylene carbonate and the like. Further, the carbon nanotubes in polymers can be oriented to be aligned by various methods known to those of ordinary skill in the art.

The technique of separating carbon nanotubes by chirality by using a chiral polymer may be further extended to a chromatography column for continuous separation. For example, carbon nanotubes wrapped in a chiral polymer may be applied to a chromatography column and then be separated by chirality. Alternatively, a suspension of exfoliated carbon nanotubes lacking a chiral agent may be applied to a chromatography column having a chiral stationary phase. In the alternative embodiments, separation by chirality is on a selective interaction of the chiral stationary phase with the various carbon nanotube chiralities.

In still further embodiments, exfoliated carbon nanotubes either with or without a wrapping chiral polymers and/or surfactants may be separated by electronic type by applying an electric potential to a solution of exfoliated carbon nanotubes. For example, exfoliated metallic carbon nanotubes will migrate toward the potential for collection and separation.

In some embodiments of the present disclosure, alternative methods for producing exfoliated carbon nanotubes not utilizing a nanocrystalline material are disclosed. In some embodiments, the methods for producing exfoliated carbon nanotubes include preparing a solution of carbon nanotubes in a superacid and filtering the solution through a filter to collect exfoliated carbon nanotubes on the filter. In some embodiments, the superacid is chlorosulfonic acid or a nitrating system.

Filtration of a superacid solution of exfoliated carbon nanotubes produces a mat of exfoliated carbon nanotubes on the filter. The mat of exfoliated carbon nanotubes may be further modified on the filter in some embodiments of the present disclosure. For example, the mat of exfoliated carbon nanotubes may be functionalized while on the filter or treated with a surfactant to maintain the carbon nanotubes in an exfoliated state. In addition, the exfoliated carbon nanotubes may be processed according to any of the methods described hereinabove for further processing of exfoliated carbon nanotubes.

The exfoliated carbon nanotubes prepared by the techniques described hereinabove are typically longer than are carbon nanotubes exfoliated using existing technology. For instance, as described previously, other separation techniques result in carbon nanotube damage and shortened carbon nanotube lengths. In certain applications, particularly those involving electrical conduction or mechanical reinforcement, shorter carbon nanotubes may not provide adequate electrical conductivity or structural reinforcement. For example, by having at least a portion of longer carbon nanotubes present with electrical devices such as energy storage device, a higher degree of connectivity at a carbon nanotube volume fraction can be obtained. Furthermore, longer carbon nanotube lengths may increase the toughness of the polymer composites over those made with shorter carbon nanotubes.

The present disclosure also relates to improved energy storage devices and particularly to ultracapacitors and batteries having components containing exfoliated carbon nanotubes. The improved energy storage devices include components such as, for example, current collectors, electrodes, insulators, electrolytes and separators containing exfoliated carbon nanotubes. The improved energy storage devices have a high energy density and power density and better discharge and charge capabilities. The improved energy storage devices have at least one of at least two electrodes containing exfoliated carbon nanotubes. The improved energy storage devices also include a dielectric medium or electrolyte, each optionally including carbon nanotubes.

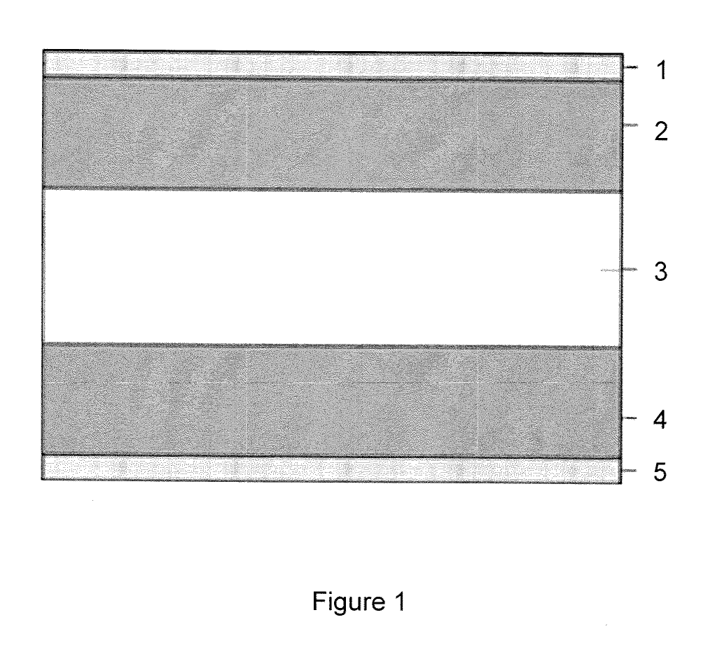

FIG. 1 shows an illustrative arrangement of the basic elements of a Faradaic capacitor. As shown in FIG. 1, current collectors 1 and 5 contact with electrodes 2 and 4, which are separated by electrode 3. In an embodiment of the present disclosure, at least one of the electrodes 2 and 4 contains exfoliated carbon nanotubes. In various embodiments, current collectors 1 and 5 can be metals such as, for example, copper and other highly conductive metals. In some embodiments, the current collectors can contain conductive exfoliated carbon nanotubes. For example, in an embodiment, the carbon nanotubes may be full length exfoliated carbon nanotubes. In some embodiments, the carbon nanotubes may be separated metallic carbon nanotubes. In various embodiments, at least one of electrodes 2 and 4 contains exfoliated carbon nanotubes.

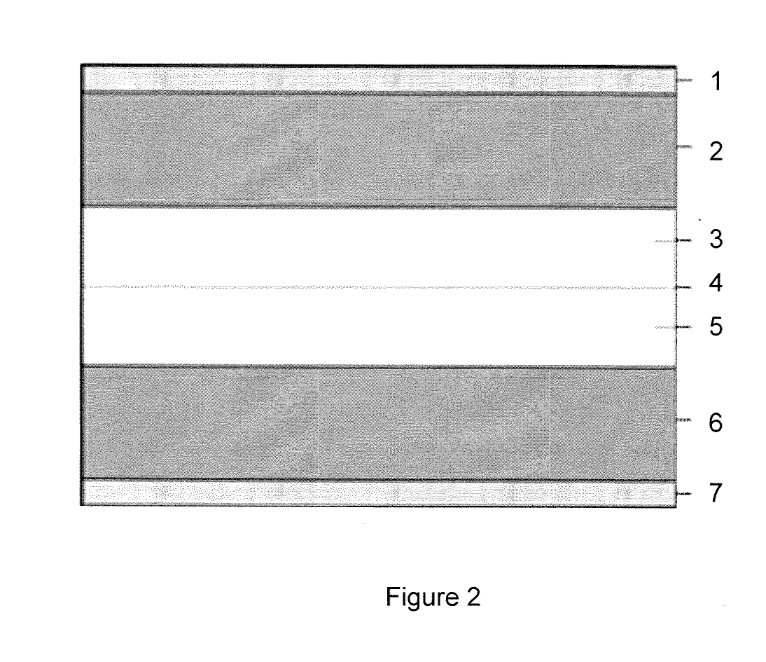

FIG. 2 shows an illustrative arrangement of the basic elements of an electric double layer capacitor. As shown in FIG. 2, current collectors 11 and 17 contact electrodes 12 and 16, and electrolytes 13 and 15 contact electrodes 12 and 16. Non-conducting separator 14 separates electrolytes 13 and 15 and is permeable to ions flowing between the electrodes 12 and 16. In some embodiments, current collectors 11 and 17 can be metals such as, for example, copper and like conductive metals. In some embodiments, current collectors 11 and 17 contain exfoliated carbon nanotubes. In some embodiments, the carbon nanotubes may be separated metallic carbon nanotubes. At least one of electrodes 12 and 16 contains exfoliated carbon nanotubes. Electrolytes 12 and 16 may be fully intermixed with the electrodes 2 and 6, or they may contact along a surface such as, for example, a plane. In various embodiments, non-conducting separator 4 may contain non-conducting carbon nanotubes. In various embodiments, the separator 4 may be made from porous polyethylene or fiberglass mats. In various embodiments, electrolytes 13 and 15 can contain exfoliated carbon nanotubes, which may be exfoliated conductive carbon nanotubes in some embodiments, conductive nanotubes in various embodiments.

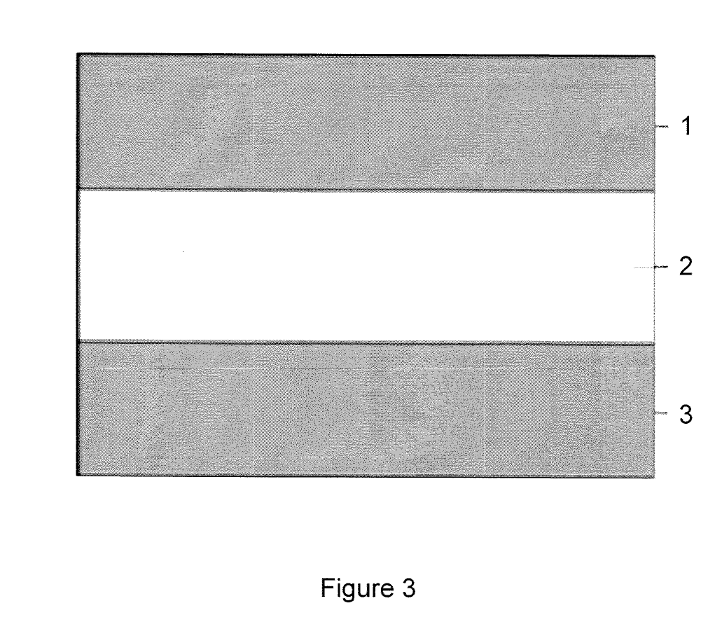

FIG. 3 shows an illustrative arrangement of the basic elements of a battery. As shown in FIG. 3, electrodes 21 and 23 contact electrolyte 22. The electrolyte 22 conveys ions between electrodes 21 and 23. In an embodiment, the ions are metal ions such as, for example, lithium ions. Hence, the present disclosure describes a lithium battery containing exfoliated carbon nanotubes. In some embodiments, at least one of the electrodes contains exfoliated carbon nanotubes. In some embodiments, the electrolyte contains exfoliated carbon nanotubes.

In various embodiments of the present disclosure, the energy storage device containing exfoliated carbon nanotubes is a battery containing at least two electrodes and an electrolyte in contact with the at least two electrodes. At least one of the electrodes contains exfoliated carbon nanotubes.

In some embodiments of the energy storage devices, the exfoliated carbon nanotubes are multi-wall carbon nanotubes. In some embodiments, the at least one electrode containing exfoliated carbon nanotubes is the anode.

In various embodiments of the energy storage devices, the electrode may contain exfoliated carbon nanotubes dispersed in a polymer or viscous liquid. After forming the electrode, in various embodiments, the may be laminated to another medium such as, for example, a dielectric or electrolyte.

In various embodiments, the electrolyte of the energy storage devices can be a solid or a fluid. Electrolytes are generally chosen to minimize internal electrical resistance. Aqueous electrolytes such as potassium hydroxide or sulfuric acid are generally employed in conventional batteries and capacitors. Due to water's low electrochemical decomposition potential of 1.24 volts, the energy density is limited with these types of electrolytes. Organic electrolytes such as, for example, organic carbonates and tetraalkylammonium salts provide good solubility and reasonable conductivity. In general, organic electrolytes have lower conductivity than aqueous electrolytes, but they can operate at higher voltages, for example, up to about 5 volts. Other electrolytes can be of a polymer-gel type such as, for example, polyurethane-lithium perchlorate, polyvinyl alcohol-KOH—H₂O and the related systems. Organic electrolytes such as, for example tetraethylammonium tetrafluoroborate and tetrabutylammonium tetrafluoroborate, can simultaneously serve as an electrolyte and surfactant for dispersing and exfoliating carbon nanotubes in embodiments where carbon nanotubes are contained in the electrolyte. Electrolyte salts may also be used for dispersing the carbon nanotubes or maintaining exfoliated carbon nanotubes in an exfoliated state.

In some embodiments of the energy storage devices, the exfoliated carbon nanotubes are modified with an electroactive material. In some embodiments, the electroactive material is a transition metal or transition metal oxide. Electroactive transition metals include, for example, Ru, Ir, W, Mo, Mn, Ni, and Co. In some embodiments, the electroactive material may be a conducting polymers such as, for example, polyaniline, polyacetylene and polyvinylpyrrole. In some embodiments, the electroactive material is a nanomaterial bound to the exfoliated carbon nanotubes. In some embodiments, the nanomaterial may be, for example, SnO₂. Li₄Ti₅O₁₂, silicon nanotubes, silicon nanoparticles and various combinations thereof.

In other various embodiments, the present disclosure describes layered structures containing exfoliated carbon nanotubes suitable for use in energy storage devices. For example, co-extrusion of liquids or melts containing exfoliated carbon nanotubes through multilayer dies or multilayer generators may be used in making the energy storage devices of the present disclosure. The resultant layered structures can be stacked and connected in series to give higher voltages in energy storage devices. In other embodiments, the components of the energy storage devices may be processed from a solution of exfoliated carbon nanotubes by solvent casting, spraying, paste spreading, compression stretching, or combinations thereof.

In some embodiments, the present disclosure also relates to an ion diffusion separator of electrical double-wall capacitors. In various embodiments, the separator contains non-metallic single-wall carbon nanotubes. In some embodiments, insulators of the energy storage devices contain non-metallic single-wall carbon nanotubes. In some embodiments, when the insulator contains carbon nanotubes, the dielectric constant of the insulator/carbon nanotube mixture is greater than that of the insulator alone.

In various embodiments, exfoliated carbon nanotubes can be aligned in forming electrodes for use in the energy storage devices. In some embodiments, the alignment may occur through melt extrusion.

In some embodiments, incorporation of exfoliated carbon nanotubes to electrodes, electrolytes or dielectrics of the present energy storage devices provides enhanced strength and ruggedness to the device. These features allow further shaping of the device for functioning under demanding environments, such as high vibration or extreme thermal cycling environments.

### Experimental Examples

The following experimental examples are included to demonstrate particular aspects of the present disclosure. It should be appreciated by those of ordinary skill in the art that the methods described in the examples that follow merely represent illustrative embodiments of the disclosure. Those of ordinary skill in the art should, in light of the present disclosure, appreciate that many changes can be made in the specific embodiments described and still obtain a like or similar result without departing from the spirit and scope of the present disclosure.

### Example A

Exfoliation of Carbon Nanotubes Using Zr(HPO₄)₂.H₂O Nanoplates and t-Butylammonium Hydroxide Surfactant. A dispersed solution of carbon nanotubes was prepared from 10 mg of multi-wall carbon nanotubes placed in 2 mL of a solution of Zr(HPO₄)₂.H₂O nanoplates and t-butylammonium hydroxide (5 wt % Zr(HPO₄)₂.H₂O: 1:0.8 ratio of Zr(HPO₄)₂.H₂O:t-butylammonium hydroxide). The solution was subsequently diluted to 30 mL and then sonicated for 2 hours. The solution was stable for at least 24 hours. An aliquot of 0.01 M KCl was added, resulting in precipitation of a quantity of exfoliated multi-wall carbon nanotubes. The precipitated fraction was removed by centrifugation. The quantity of isolated nanotubes was approximately 1/10 the mass of carbon nanotubes originally suspended. The filtrate was treated with another aliquot of 0.01 M KCl, resulting in a second precipitation of multi-wall carbon nanotubes. The precipitation/centrifugation process was repeated until substantially all nanotubes had been precipitated from the suspension.

### Example B

Exfoliation of Carbon Nanotubes Using Zr(HPO₄)₂.H₂O Nanoplates of Varying Sizes. The experimental procedure described in Example A hereinabove was repeated, except the nanoplate size was about 1/10 the length of the longest carbon nanotube present in the sample. After removal of the first precipitation fraction following addition of 0.01 M KCl, a second quantity of nanoplates of a different size was added. The second quantity of nanoplates fractionated a second quantity of nanotubes following addition of 0.01 M KCl. The second precipitation fraction of nanotubes had a different length distribution than did the first precipitation fraction. The precipitation centrifugation process was repeated with progressively larger nanoplates until substantially all nanotubes had been precipitated from the suspension.

### Example C

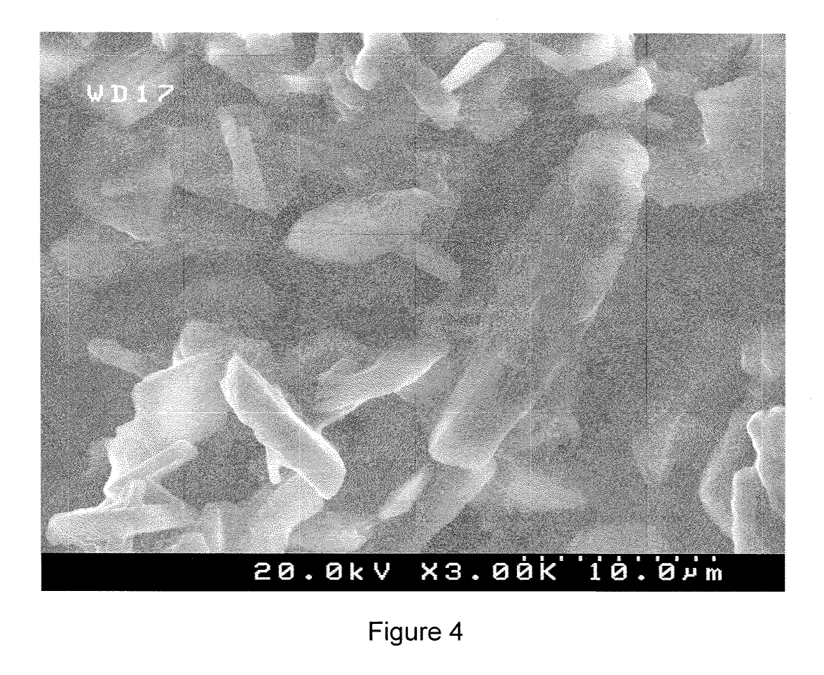

Synthesis of Hydroxyapatite Plates. Hydroxyapatite nanoplates of controlled sizes were synthesized by dissolving 10 g of hydroxyapatite (Sigma Aldridge reagent grade) in 400 mL of dilute nitric acid (pH=2) at room temperature, followed by very slow dropwise addition of 48 mL of 1% v/v ammonium hydroxide. Crystals collected at pH=4 and pH=5 were found by microscopy to be plates having an aspect ratio about 7 to 8 and a diameter ranging between 3-15 μm. FIG. 4 shows an illustrative electron micrograph of hydroxyapatite plates of 3-15 μm diameter. Increasing the addition rate of the 1% v/v ammonium hydroxide reduced the average HAp plate size.

### Example D

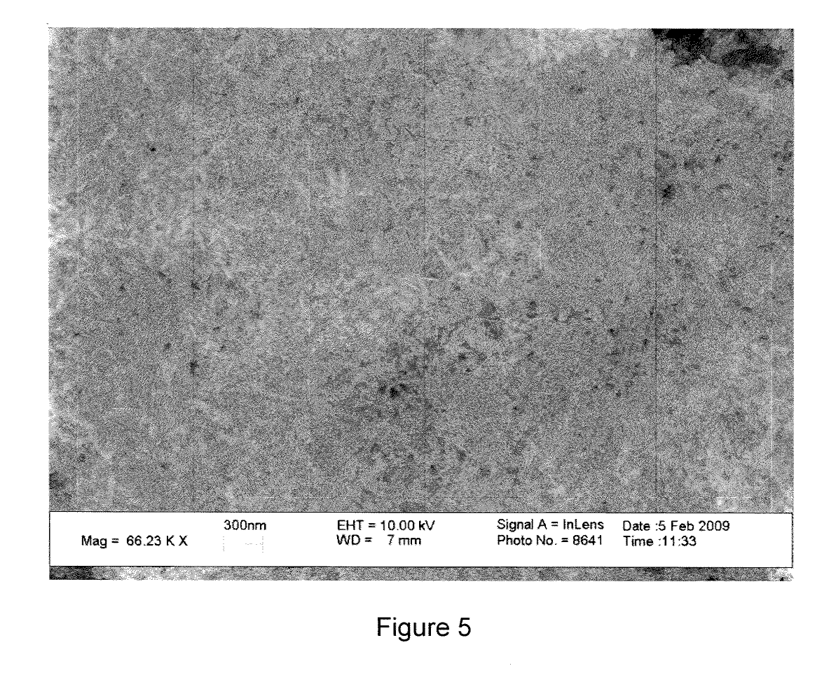

Synthesis of Hydroxyapatite Nanorods. 2 g of hydroxyapatite was first dissolved in 40 mL of dilute nitric acid (pH=2) containing a 3:1 ethanol:water ratio. The mixture was then quenched into 80 mL of 5 vol % ammonium hydroxide, also in a 3:1 ethanol:water ratio. The resultant pH was 8.5. A milky, jelly-like precipitate resulted. The resulting mixture containing the precipitate was then heated at between 70° C. and 80° C. on a magnetic stirrer hotplate for 24 hours. Thereafter, hydroxyapatite crystals were filtered, washed with deionized water and dried. Electron microscopy showed that hydroxyapatite nanorods having an aspect ratio of about 25 and lengths between 100-200 nm were formed. FIG. 5 shows an electron micrograph of hydroxyapatite nanorods having 100-200 nm lengths.

### Example E

Exfoliation of Carbon Nanotubes Using Hydroxyapatite. 0.5142 g hydroxyapatite nanorods were added to 50 mL water and 0.8280 g t-butylammonium hydroxide (Sigma Aldrich reagent grade; TBAH; 1:1 molar ratio of hydroxyapatite:TBAH). The resultant mixture was sonicated for one hour at 25° C. then diluted with deionized water to give a 0.2 wt % solution based on hydroxyapatite content. Multi-wall carbon nanotubes (CNano Ltd;) were received as a powder that contained highly entangled bundles having a grain size of 1-10 μm in diameter. The lengths of the individual multi-wall carbon nanotubes were found to be in excess of 1 μm, and the diameters were found to be 10-20 nm.

1 g of multi-wall carbon nanotubes was added to 50 mL of a mixture of concentrated sulfuric and nitric acid in a 3:1 volume ratio. The mixture was placed in a sonicator bath (Branson sonicator, model 250) and oxidized for two hours while sonicating at temperature of 25-35° C. The mixture was then filtered using a polyvinylidene fluoride microporous filter (5 μm pore size), followed by washing of the resultant solid with deionized water until the pH of the filtrate was 4.5. The oxidized multi-wall carbon nanotubes were then dried in vacuo for 2 hours at 80° C.

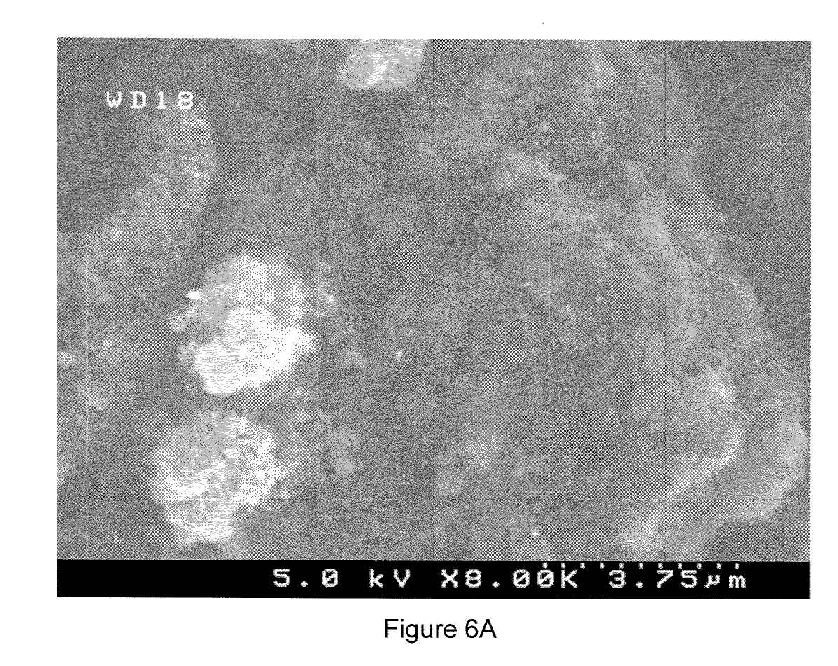

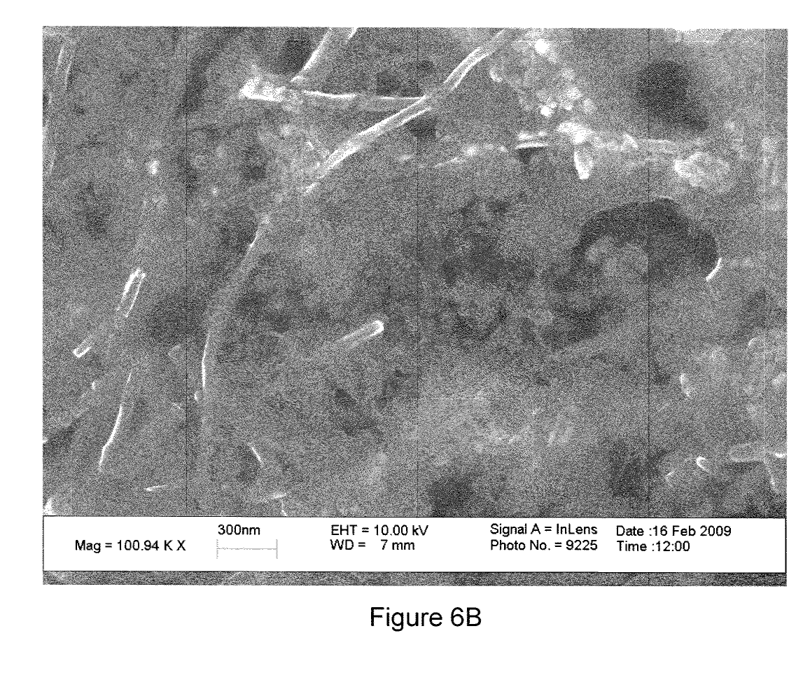

Samples were prepared by adding the dried multi-wall carbon nanotubes to the hydroxyapatite/TBAH solution prepared above to give carbon nanotube: hydroxyapatite weight ratios of 1:1, 1:2, 1:3, 1:4 and 1:5. The mixture was sonicated at room temperature for 2 hours and then left for 24 hours. At weight ratio of 1:1, a portion of the multi-wall carbon nanotubes settled out as agglomerated particles. At a 1:2 weight ratio the solution had a few multi-wall carbon nanotube particles present after 24 hours. All higher weight ratios examined gave stable dispersions for at least 24 hrs. A control experiment at a weight ratio of 1:3 multi-wall carbon nanotubes:TBAH with no hydroxyapatite present showed mostly aggregated carbon nanotubes settling after 24 hours. FIG. 6A shows an electron micrograph of as-received multi-wall carbon nanotubes, and FIG. 6B shows multi-wall carbon nanotubes exfoliated using hydroxyapatite nanorods.

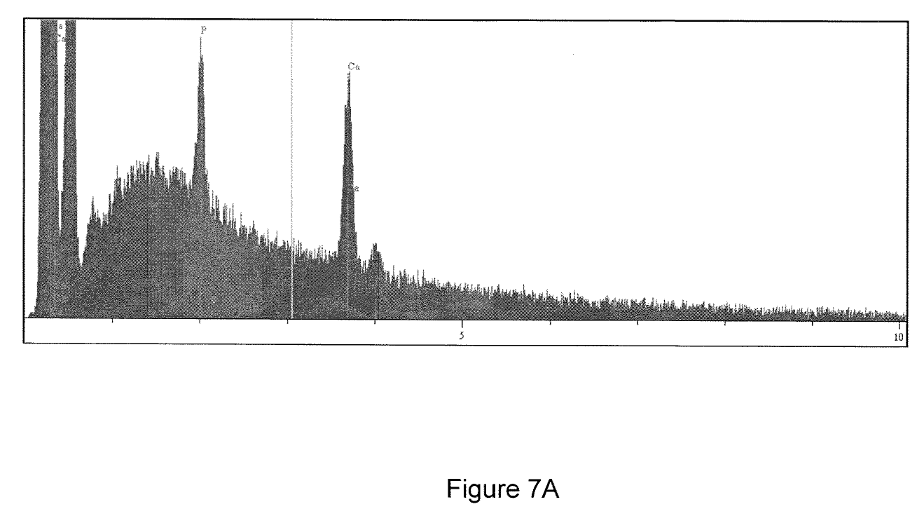

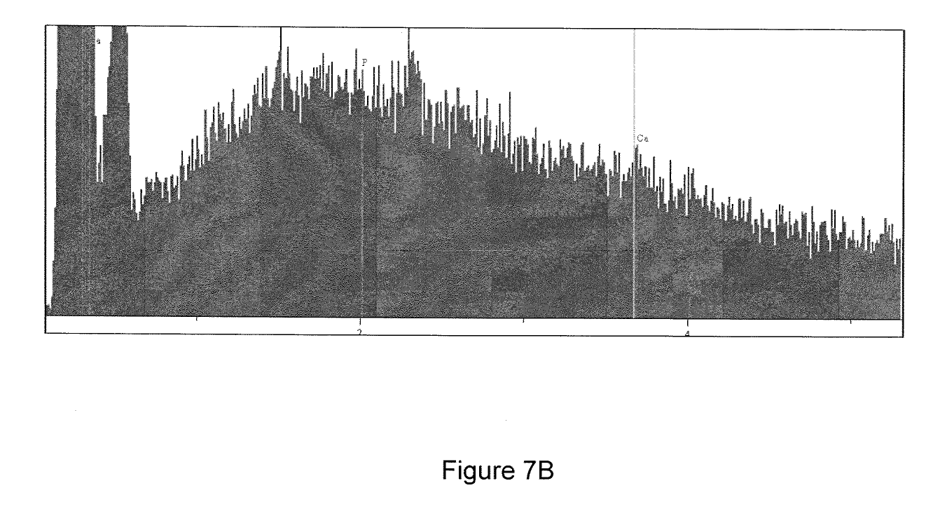

The precipitated exfoliated multi-wall carbon nanotubes contained residual hydroxyapaptite as evidenced by energy-dispersive X-ray spectroscopy. FIG. 7A shows an EDX spectrum of precipitated exfoliated multi-wall carbon nanotubes. As shown in the EDX spectrum, strong Ca and P signals indicated the presence of hydroxyapatite. The precipitated multi-wall carbon nanotubes were subsequently washed with 50 mL of 1 N nitric acid, followed by 250 mL of deionized water, which removed substantially all the hydroxyapatite as evidenced by EDX. FIG. 7B shows an EDX spectrum of precipitated exfoliated multi-wall carbon nanotubes after acid washing. In contrast, the exfoliated multi-wall carbon nanotubes of Example 1 contained residual Zr(HPO₄)₂.H₂O, which could not be removed by washing with acids such as nitric, hydrochloric or sulfuric acids.

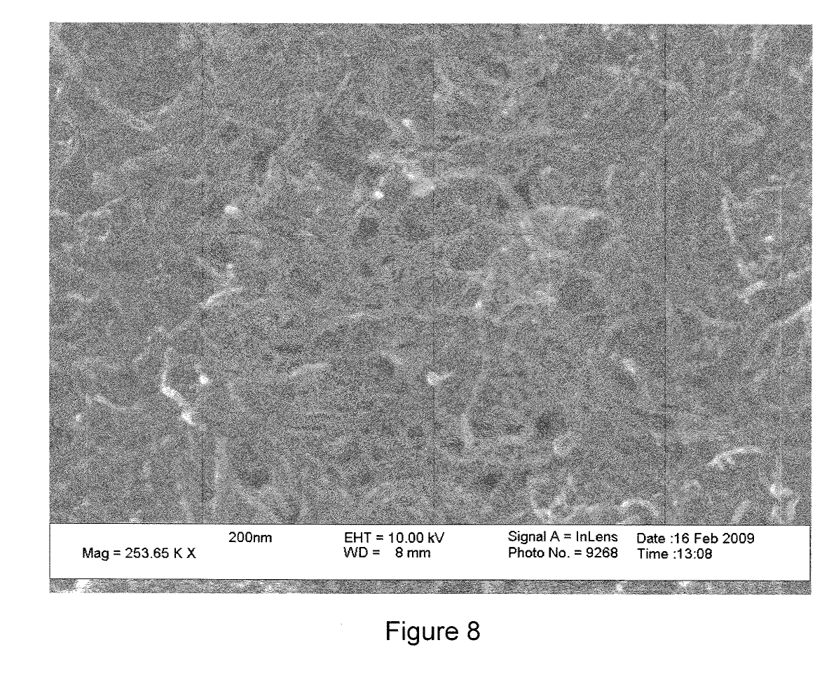

Unentangled multi-wall carbon nanotubes were obtained after exfoliation, precipitation and washing. FIG. 8 shows an electron micrograph of the exfoliated multi-wall carbon nanotubes after precipitation and washing. Exfoliation of the multi-wall carbon nanotubes could be conducted equivalently using hydroxyapatite plates.

### Example F

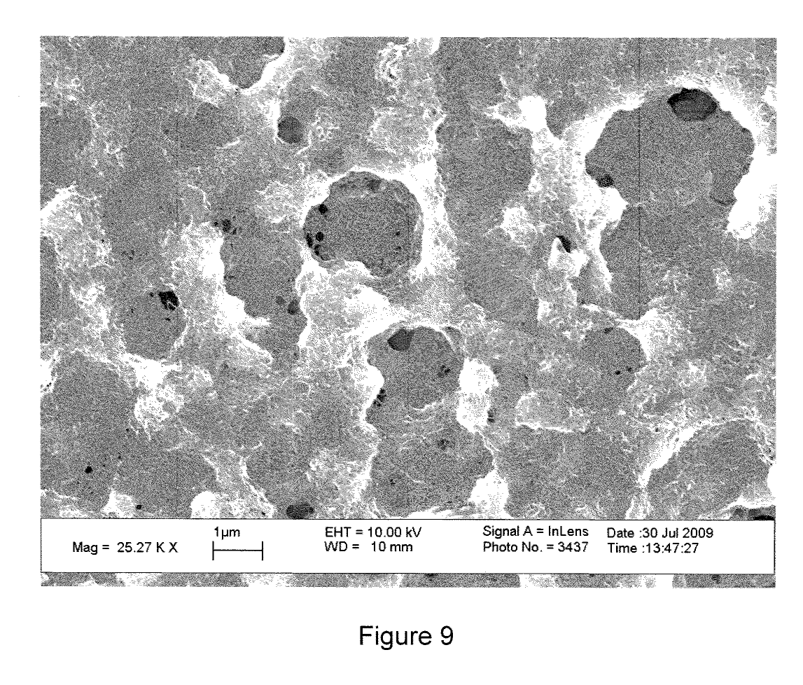

Exfoliation of Carbon Nanotubes Using Concentrated Acid Solutions. 40 mg of multi-wall carbon nanotubes were added to 40 mL of a 3:1 sulfuric:nitric acid mixture and sonicated for 60 minutes at 25° C. A drop of the mixture was placed on a PVDF filter and allowed to dry. FIG. 9 shows an electron micrograph of exfoliated carbon nanotubes obtained from 3:1 H₂SO₄:HNO₃. As shown in FIG. 9, exfoliation was maintained after removal of the acid by drying.

### Example G

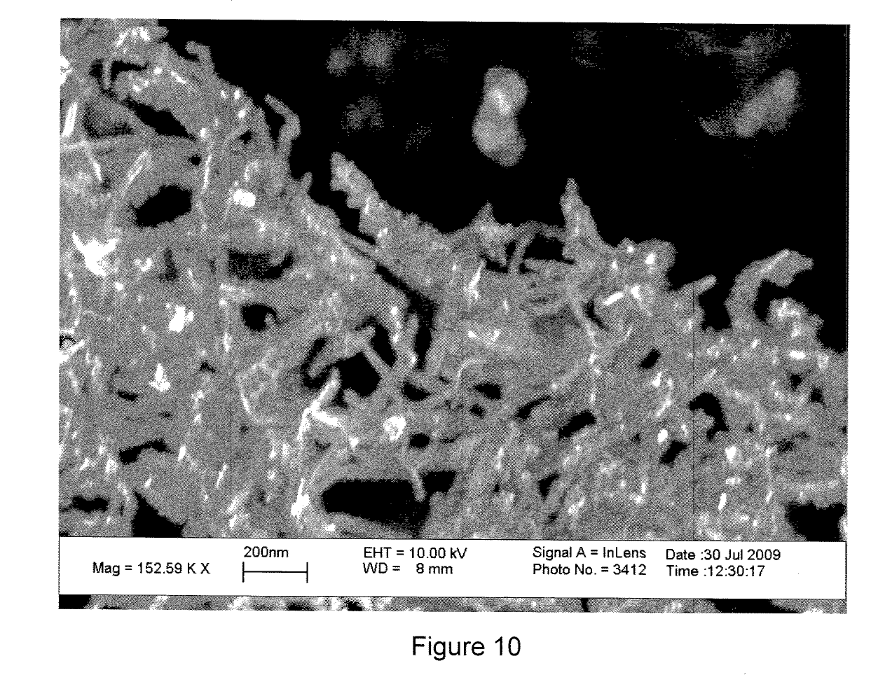

Exfoliation of Carbon Nanotubes Using Concentrated Acid Solutions, Followed by Surfactant Addition. A 1% by weight double-wall carbon nanotube solution in 3:1 sulfuric:nitric acid was oxidized for 2 hours as described previously. After filtering the concentrated acid solution to immobilize the double wall carbon nanotubes, the immobilized carbon nanotubes were washed with deionized water until the washings were pH=4.5. While still wet, the PVDF filter paper and the double-wall carbon nanotubes were sonicated for 30 minutes with a 0.2% by weight sodium dodecyl sulfate (SDS) solution in deionized water such that the weight of double-wall carbon nanotubes to SDS was 1:3. The mixture was stable for at least 24 hours. A drop of the mixture was placed on a carbon tape and dried for examination by electron microscopy, which showed exfoliated carbon nanotubes. FIG. 10 shows an electron micrograph of exfoliated double-wall carbon nanotubes following acid exfoliation and treatment with sodium dodecyl sulfate.

### Example H

Epoxy Composite Containing Exfoliated Carbon Nanotubes. 5 mg of oxidized multi-wall carbon nanotubes were placed in 10 mL of tetraethylenetetramine (TETA), and various additions of sodium dodecylsulfate (SDS) were added such that the weight ratio of multi-wall carbon nanotubes to SDS was 5, 2.5, 1, and 0.33 to 1. The mixture was sonicated at 30° C. for 30 minutes and allowed to stand. After 7 days the 1:1 and 1:0.33 ratio was seen to be stable toward precipitation.

49 g of Bisphenol F epoxy was admixed with 0.242 g of oxidized multi-wall carbon nanotubes and sonicated for 10 minutes at 60° C. The mixture was cooled to 25° C. and then degassed for 10 minutes at 25 inches Hg. 7 g of TETA containing 0.5% wt oxidized multi-wall carbon nanotubes and 0.5% wt. SDS was sonicated and degassed separately as above. The two degassed mixtures were then carefully mixed and poured into a mold. The mold was cured for 2 hours at 100° C. Controls were prepared as above without carbon nanotubes (control 1) and with as-received multi-wall carbon nanotubes (control 2).

Table 3 shows the mechanical strength improvement in the epoxy composite containing exfoliated multi-wall carbon nanotubes. Kg is a the maximum stress before failure on tensile testing a notched specimen at 0.01.min initial strain rate. Relative fatigue lifetime improvement is the lifetime of the notched specimen counted as the number of cycles to failure at 1 Hz, at about 16.7 MPa maximum tensile stress with stress amplitude of 0.1 (stress minimum/stress maximum.)

TABLE 3
Mechanical Properties of Carbon Nanotube Composites

| | | Relative fatigue lifetime |
|---|---|---|
| Material | Relative Kq improvement | improvement |
| Control 1 | 1 | 1 |
| Control 2 | 1.2 | 1.1 |
| Example 1 | 1.5 | 4.7 |

### Example I

Capacitor Containing Exfoliated Multi-Wall Carbon Nanotubes. Control 1: 10 g of poly(ethylene oxide) (PEO; 1500 molecular weight) was melted, and 1 mL of 4 N potassium hydroxide added to make the electrolyte. 1 wt % of as-received multi-wall carbon nanotubes were added to the electrolyte mixture and sonicated for 15 minutes in a sonicator bath. Approximately 2.1 g of the mixture was poured into one part of a polystyrene petri dish 6 cm in diameter with a strip of copper adhered as the current collector. A piece of clean writing paper was then placed on the molten liquid electrolyte, and 2 g of the electrolyte was poured on to the paper, taking care not to weep at the edges. The other side of the petri dish with a copper strip adhered was then inserted to make a capacitor. After cooling to room temperature for 15 minutes the capacitance was measured using an HP 4282A capacitance meter. The measured capacitance was 0.0645 microfarads. Control 2: Control 2 was prepared as for control 1, except as-received graphene (Rice University) was substituted for the multi-wall carbon nanotubes. The measured capacitance was 0.176 microfarads. Exfoliated carbon nanotube capacitor: The capacitor was prepared as for control 1, except oxidized multi-wall carbon nanotubes were used in place of as-received multi-wall carbon nanotubes. The measured capacitance was 0.904 microfarads, a 14-fold improvement over control 1 and a 5.1-fold improvement over control 2.

### Example J

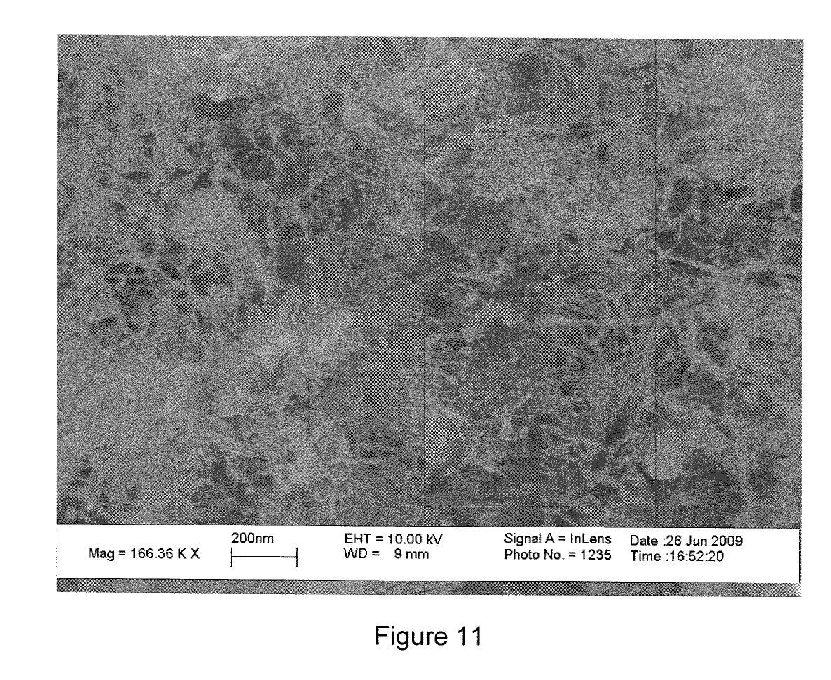

Exfoliated Carbon Nanotubes Decorated with Copper Nanoparticles. 102 mg of oxidized multi-wall carbon nanotubes were added to 100 mg copper sulfate, 640 mg sodium EDTA, 15 mg of polyethylene glycol, 568 mg of sodium sulfate and 60 mL of deionized water. The mixture was sonicated for 10 minutes and then heated to 40° C. 3 mL of formaldehyde (37% solution) and 500 mg of sodium hydroxide were added to bring the pH to 12.2. The mixture was stirred for 30 minutes at 85° C. and then filtered using a 5 micron PVDF filter and washed with 200 mL of deionized water. FIG. 11 shows an electron micrograph of exfoliated carbon nanotubes decorated with copper oxide nanoparticles obtained from the mixture.

From the foregoing description, one of ordinary skill in the art can easily ascertain the essential characteristics of this disclosure, and without departing from the spirit and scope thereof, can make various changes and modifications to adapt the disclosure to various usages and conditions. The embodiments described hereinabove are meant to be illustrative only and should not be taken as limiting of the scope of the disclosure, which is defined in the following claims.

We claim:

1. A plurality of carbon nanotubes comprising carbon nanotube fibers having an aspect ratio of from about 25 to about 500, and a oxidation level of from about 3 weight % to about 15 weight %.

2. The fibers of claim 1 wherein a neutralized water treatment of the fibers results in a pH of from about 3 to about 9, preferably from about 4 to about 8.

3. The fibers of claim 1 wherein the oxidation species comprises carboxylic acid or derivative carboxylate groups.

4. The fibers of claim 1 wherein the fibers are discrete individual fibers, not entangled as a mass.

5. A plurality of carbon nanotubes comprising discrete carbon nanotube fibers having an aspect ratio of from about 25 to about 250 and a oxidation level of from about 3 wt % to about 15 wt %, wherein the fibers are mixed, blended, sonicated, or a combination step thereof, with at least one epoxy resin to form an epoxy/nanotube composite.

6. A plurality of carbon nanotubes comprising discrete carbon nanotube fibers having an aspect ratio of from about 25 to about 250 and a oxidation level of from about 3 wt % to about 15 wt %, wherein the fibers are mixed, blended, sonicated, or a combination step thereof, with at least one rubber compound to form a rubber/nanotube composite.

7. The fibers of claim 1 comprising a residual metal concentration of less than about 1000 ppm.

8. The fibers of claim 1 comprising a residual metal concentration of less than about 100 ppm.

9. The fibers of claim 1 comprising open ended carbon nanotube fibers.

10. The fibers of claim 1, wherein a mat of said fibers are electrically conductive.

11. The fibers of claim 10, wherein said mat has an electrical conductivity of a east 0.1 Siemens/cm and as high as about 100 Siemens/cm.

12. The fibers of claim 1, wherein said fibers have an average diameter of from about 0.6 nanometers to about 30 nanometers.

13. The fibers of claim 1, wherein said fibers have an average length of from about 50 nanometers to about 10000 nanometers.

14. A method for preparing carbon nanotube fibers, said method comprising:
a) suspending entangled non-discrete multi-wall carbon nanotube fibers in an acidic solution for a time period;
b) optionally agitating said composition;
c) sonically treating said suspended nanotube fiber composition to form discrete carbon nanotube fibers; and
d) isolating the resultant discrete carbon nanotube fibers from the composition prior to further treatment using solid/liquid separations, wherein said separations comprise, filtration and centrifugation.

15. The method of claim 14, wherein the acidic solution comprises a solution of sulfuric acid and nitric acid.

16. The method of claim 15, wherein the nitric acid is present in a dry basis of from about 10 weight % to about 50 weight %, preferably from about 15 weight % to about 30 weight %.

17. The method of claim 14, wherein the sonic treatment is performed at an energy input of from about 200 to about 600 Joules/gram of suspended composition.

18. The method of claim 14, wherein the non-discrete carbon nanotube fibers are present in a concentration of from greater than zero to less than about 4 weight percent of the suspended nanotube fiber composition.

19. The method of claim 14, wherein the suspended discrete nanotube fiber composition in the acidic solution is controlled at a specific temperature environment.

20. The method of claim 19, wherein the specific temperature environment is from about 15 to 65° C., preferably from about 25° to about 35° C.

21. The method of claim 14, wherein said method comprises a batch, semi-batch, or continuous method.

22. The method of claim 14, wherein the composition is in contact with the acidic solution from about 1 hour to about 5 hours.

23. The method of claim 14, wherein said isolated resultant discrete carbon nanotube fibers from the composition prior to further treatment comprises at least about 10 weight percent water.

24. The fibers of claim 1, wherein the fibers are at least partially surface modified or coated with at least one surfactant.

25. The fibers of claim 1, wherein the fibers are completely surface modified or coated.

26. The fibers of claim 1, wherein the fibers are at least partially surface modified or coated with at least one modifier.

27. The fibers of claim 1, wherein the fibers are completely surface modified or coated.

28. The fibers of claim 24, wherein the surfactant or modifier is hydrogen bonded, covalently bonded, or ionically bonded to the carbon nanotube fibers.

29. The fibers of claim 24, wherein said coating is substantially uniform.

30. The fibers of claim 24, wherein the fibers are further mixed, blended, sonicated, or a combination method thereof, with at least one elastomer to form an elastomer nanotube fiber composition.

31. The fibers of claim 30 wherein the elastomers comprises natural rubber, synthetic rubber, or rubber compounds comprising fillers of carbon or silicon compounds, and wherein a fiber surface modifier or the surfactant is chemically, physically, or both, bonded to the elastomer, an isolated fibers, or any fillers present.

32. The elastomer nanotube fiber composition of claim 30 wherein said modifier or surfactant is chemically bonded to said elastomer, said nanotube fiber, or both.

33. The fibers of claim 24, wherein the fibers are further mixed, blended, sonicated, or a combination method thereof, with at least one other material to form a material/nanotube fiber composition.

34. The material nanotube fiber composition of claim 32, wherein said modifier or surfactant is chemically bonded to said material or said nanotube fiber.

35. The fibers of claim 24, wherein the fibers are further mixed, blended, sonicated, or a combination method thereof, with at least one epoxy to form an epoxy/nanotube fiber composition.

36. The epoxy/nanotube fiber composition of claim 35, wherein said modifier or surfactant is chemically bonded to said epoxy, said nanotube fiber, or both.

37. The epoxy/nanotube fiber composition of claim 35, wherein said composition has a fatigue crack failure resistance of at least 2 to about 20 times the fatigue crack failure resistance of the epoxy tested without carbon nanotubes.

38. The epoxy/nanotube fiber composition of claim 35, w herein said composition has a coefficient of expansion in at least one dimension of at least ⅔ to ⅓ that of the epoxy tested without carbon nanotubes in the same dimension.

39. The elastomer/nanotube fiber composition of claim 30, wherein said composition has a fatigue crack failure resistance of at least 2 to about 20 times the fatigue crack failure resistance of the elastomer tested without carbon nanotubes.

40. A material-nanocomposite fiber composition of claim 32 bonded to a substrate, wherein said composition has an adhesive or cohesive strength of at least two times greater that of the material without carbon nanotubes tested similarly.

41. An elastomer-nanocomposite fiber composition of claim 30 bonded to a substrate, wherein said composition has an adhesive or cohesive strength of at least two times greater that of the elastomer without carbon nanotubes tested similarly.

42. An epoxy-nanocomposite fiber composition of claim 35 bonded to a substrate, wherein said composition has an adhesive or cohesive strength of at least two times greater that of the epoxy without carbon nanotubes tested similarly.

43. An epoxy-nanocomposite fiber composition of claim 35 bonded to a substrate, wherein said composition has an adhesive or cohesive strength of at least two times greater that of the epoxy without carbon nanotubes tested similarly.

44. The fibers of claim 24 wherein the fibers are further mixed, blended, sonicated, or a combination method thereof, with at least one elastomer and an inorganic nanoplate to form an elastomer nanotube fiber and nanoplate composition.

45. The elastomer nanotube fiber and nanoplate composition of claim 30, wherein the carbon nanotube and/or nanoplate is chemically bonded to said elastomer.

46. A cyano-acrylate containing material containing fibers of claim 24 bonded to a substrate wherein said composition has an adhesive or cohesive strength of at least two times greater that of the cyano-acrylate containing material without carbon nanotubes tested similarly.

47. The carbon nanotube fibers of claim 1 comprising single, double, or multi-wall fibers.
